(19) United States
(12) Patent Application Publication
(10) Pub. No.: US 2024/0165261 A1
(43) Pub. Date: May 23, 2024

(54) MEANS AND METHOD FOR CYTOSOLIC DELIVERY

(71) Applicant: Universiteit Gent, Gent (BE)

(72) Inventors: Stefaan DE SMEDT, Mariakerkerke (BE); Koen RAEMDONCK, Gent (BE)

(21) Appl. No.: 18/552,838

(22) PCT Filed: Mar. 28, 2022
(86) PCT No.: PCT/EP2022/058092

§ 371 Date: Sep. 27, 2023

(30) Foreign Application Priority Data
Mar. 29, 2021 (EP) 21165687.1

(51) Int. Cl. A61K 47/69; A61K 47/61

(52) U.S. Cl. CPC A61K 47/6903; A61K 47/61

(57) ABSTRACT

The present invention is related to the field of intracellular delivery of membrane-impermeable materials. It provides compositions enabling the delivery of such membrane-impermeable materials into cells, applicable for both in vitro, in vivo and ex vivo delivery applications, as well as the use thereof in methods of cytosolic delivery of membrane-impermeable materials. The compositions and methods are particularly useful in biological research, diagnostic methods and the development of cell-based therapies.

### FIELD OF THE INVENTION

The present invention is related to the field of intracellular delivery of membrane-impermeable materials. It provides compositions enabling the delivery of such membrane-impermeable materials into cells, applicable for both in vitro, in vivo and ex vivo delivery applications, as well as the use thereof in methods of cytosolic delivery of membrane-impermeable materials. The compositions and methods are particularly useful in biological research, diagnostic methods, local drug delivery and the development of cell-based therapies.

### BACKGROUND TO THE INVENTION

Intracellular delivery techniques are designed to introduce otherwise cell-impermeable molecules (e.g. small molecules, peptides, proteins, nucleic acids, . . . ) into the cytoplasm, enabling us to guide cell fate, probe cell function and reprogram cell behaviour. As such, intracellular delivery not only contributes to our fundamental understanding of cell biology, but also allows to create new or improve existing therapeutic strategies.

A wide variety of cargo and target cell types can be envisioned, both for in vitro, in vivo and ex vivo applications (1,2). In particular, ex vivo cell engineering entails specific needs regarding intracellular delivery approaches (2). Decades of clinical experience have shown that ex vivo culturing of cells comes with risks of inducing undesired geno- and phenotypic alterations, for instance loss of cytokine production or even exhaustion of the proliferative potential of adoptive cell therapies. As such, minimizing the time in culture is vital, requiring high-throughput delivery techniques that offer high delivery efficiencies while maintaining a high cell viability. In addition, many applications (e.g. differentiation of stem cells) require an ideal combination of small molecules and macromolecular cargo to be delivered, demanding highly flexible delivery techniques (1). Finally, with regard to fundamental research and even more so the scale-up of cell therapies, cost-effective and straightforward delivery methods are needed.

Intracellular delivery techniques can be divided into two major categories, i.e. membrane-disruption and carrier-based delivery methods. Membrane disruption-mediated delivery typically requires an external physical (e.g. mechanical, electrical, thermal, optical) or chemical (e.g. oxidants, pore-forming agents) trigger to transiently permeabilize the cell membrane. These methods generally offer great flexibility, allowing efficient cytosolic delivery of cargo with divergent physicochemical properties in a wide variety of cell types. However, the need for external stimuli generally requires specialized instrumentation. In addition, the generation of membrane defects is often associated with a substantial loss of cell viability and the induction of undesirable cellular stress responses.

Carrier-based delivery relies on nanoparticles to package and deliver membrane-impermeable cargo into cells. In general, both viral and non-viral delivery nanocarriers can be distinguished. Due to their high efficiency, viral vectors belong to the most clinically advanced delivery carriers for nucleic acid delivery. Non-viral nanocarriers typically make use of (semi-)synthetic materials (e.g. polymers, lipids, inorganic nanomaterials) that either electrostatically complex or physically entrap their (charged) cargo. The ability of non-viral carriers to protect cargo from degradation and target specific tissues, as well as their scalability makes them attractive options for both in vivo and ex vivo delivery applications (3-5). Nevertheless, cargo encapsulation is generally dependent on the physicochemical properties of both the carrier and the cargo, limiting cargo flexibility compared to membrane disruption-based methodologies. As for most viruses, also non-viral carriers typically enter cells via one or more endocytic pathways depending on their physicochemical properties and the type of cell surface interaction. These pathways are often ill-defined and cell type dependent, complicating widespread use. Furthermore, since endosomal content is prone to rapid recycling towards the cell surface or lysosomal degradation, efficient endosomal or lysosomal escape strategies are required for cytosolic cargo delivery. Multiple escape mechanisms have been evaluated (e.g. based on endosomal membrane fusion or disruption), but to date remain largely inefficient. In addition to these hurdles, sufficient and timely cargo release from the nanocarrier following endocytosis remains a major bottleneck limiting intracellular delivery efficiency.

Polycationic materials, such as cationic liposomes and polymers, have been extensively researched for complexation of polyanionic molecules such as nucleic acids into nanoparticles (6). Furthermore, they have been shown to internalize more efficiently into cells than their negatively charged counterparts, owing to the electrostatic interaction with the negatively charged cell membrane (7). Notably, multiple studies have shown that commonly used cationic polymers (e.g. DEAE-dextran, polyethyleneimine, PAMAM-dendrimers) and cationic nanoparticles (e.g. surface-modified mesoporous silica nanoparticles and gold nanoparticles) can also induce membrane disruption events such as increased membrane fluidity, membrane thinning and the formation of nanoscale holes, even at non-toxic concentrations. Remarkably, studies using supported lipid bilayers as model membranes revealed that localized membrane thinning events typically precede the complete removal of lipids, eventually resulting in membrane pores with an estimated average size of 15-40 nm in diameter. In addition, theoretical studies have shown these pore formation events to be thermodynamically feasible. Practical evidence for increased cell permeability was mainly provided by showing the influx of small membrane-impermeable molecules (e.g. fluorescent dyes and ions) in viable, polycation-treated cells (7, 8, 9, 10).

Despite the breadth of available intracellular delivery tools, existing protocols are often suboptimal and alternative approaches that merge delivery efficiency with both biocompatibility, as well as applicability, remain highly sought after.

We have demonstrated in the present invention that supramolecular cationic materials, and in particular supramolecular cationic nanoparticles not only function as carrier-mediated delivery strategies, but can also be positioned as membrane-disruptive agents to enable direct cytosolic delivery of membrane-impermeable compounds, i.e. in enabling a crossing of the cell membrane without the supramolecular cationic nanoparticles functioning as a carrier for said membrane-impermeable compounds. As such, important bottlenecks such as inefficient cargo decomplexation and endosomal escape can be circumvented. We show that crosslinked cationic hydrogel nanoparticles, in contrast to exemplary cationic polymers, can effectively permeabilize the plasma membrane of different cell types without excessive cell damage, allowing highly efficient cytosolic delivery of several classes of molecules based on the absence of an electrostatic attraction between said membrane-impermeable compounds and the supramolecular cationic nanoparticles.

### SUMMARY OF THE INVENTION

In a first aspect the present invention provides a method for delivery of cell-impermeable molecules across a cell membrane, said method comprising contacting cells with cationic cell membrane permeabilizing materials, such as crosslinked cationic polymers, cationic hydrogels, and/or cationic nanoparticles, hereinafter commonly referred to as supramolecular polycationic materials, in the presence of the cell-impermeable molecules to be delivered across the cell membrane, and wherein the cell-impermeable molecules and polycationic materials are independently supplied either simultaneous (co-delivery) or sequentially to the cells.

In said method, the supramolecular polycationic materials disrupt the membrane of the cell and accordingly act as permeabilizing agents. Different to the typical application of said polycationic materials, in the methods of the present invention, the polycationic materials do not act as carriers (e.g. by complexation or encapsulation) for the cell-impermeable molecules to be delivered across the cell membrane. There is no interaction between the cell-impermeable molecules and the polycationic materials. As a consequence, the cell-impermeable molecules are free in suspension/solution/medium. This implies that within the method according to the invention the cell-impermeable molecules and polycationic materials are independently supplied either simultaneous (co-delivery) or sequentially. Expressed differently, in the methods according to the invention the supramolecular polycationic materials and the cell-impermeable molecules do not interact with one another. The supramolecular polycationic materials create pores in the membrane, through which the otherwise cell-impermeable molecules can passively cross the cell membrane.

It is accordingly an embodiment of the present invention to provide a method for delivery of cell-impermeable molecules across a cell membrane, said method comprising contacting cells with supramolecular polycationic materials, in the presence of the cell-impermeable molecules to be delivered across the cell membrane, characterized in that the cell-impermeable molecules and supramolecular polycationic materials are independently supplied either simultaneous (co-delivery) or sequentially to the cells, i.e. characterized in that the cell-impermeable molecules and supramolecular polycationic materials do not interact with one another.

Relying on the membrane permeabilizing effect of the supramolecular polycationic materials, without an interaction with the cargo to be delivered across the membrane, the method works best with cell-impermeable molecules that in themselves show no interaction with the supramolecular polycationic materials, such as neutral or cationic cell-impermeable molecules with respect to the polycationic materials when supplied to the cell. Expressed differently, and as already mentioned herein before, using the supramolecular polycationic materials as herein provided, the transport across the cell membrane is based on the absence of an electrostatic attraction between the cargo to be delivered across the cell membrane and the supramolecular polycationic materials. It accordingly works best with neutral or cationic cell-impermeable molecules with respect to the polycationic materials when supplied to the cell.

Hence in another embodiment the present invention provides a method for delivery of cell-impermeable molecules across a cell membrane and/or into the cytosol of a cell, said method comprising contacting cells with supramolecular polycationic materials, in the presence of the cell-impermeable molecules to be delivered across the cell membrane, characterized in that the cell-impermeable molecules are neutral or cationic cell-impermeable molecules with respect to the supramolecular polycationic materials when supplied to the cell. In a particular embodiment said neutral or cationic cell-impermeable molecules are independently supplied, either simultaneous (co-delivery) or sequentially, with the membrane permeabilizing supramolecular polycationic materials. In a particular embodiment the neutral or cationic cell-impermeable molecules and the supramolecular polycationic materials are sequentially supplied, with the membrane permeabilizing supramolecular polycationic materials prior to the neutral or cationic cell-impermeable molecules.

In one embodiment the supramolecular polycationic materials used in the methods according to the invention are selected from crosslinked cationic polymers and cationic nanoparticles or combinations thereof. In a preferred embodiment the supramolecular polycationic materials are selected from crosslinked cationic polymers and crosslinked cationic nanoparticles; more in particular crosslinked cationic hydrogel nanoparticles; even more in particular crosslinked dextran nanogels such as dextran methacrylate (MA)-co-TMAEMA nanogels, and the like.

Best results were obtained with crosslinked dextran nanogels having a Degree of Methacrylate Substitution (DS; number of methacrylates per 100 glucopyranose units) of at least 2.5, in particular at least 3.4, more in particular 4.7, even more in particular at least 5.9, and a cationic charge with a zeta potential of at least 5 mV, in particular at least 6, 7, 8, 9 or 10 mV, more in particular at least 11 mV, even more in particular at least 16 mV; and in a most particular embodiment with a zeta potential of at least 21 mV. Thus in one embodiment the present invention provides a method for delivery of cell-impermeable molecules across a cell membrane, said method comprising contacting cells with crosslinked dextran nanogels having a DS of at least 2.5; in particular a DS of at least 3.0; in particular a DS of at least 3.4, more in particular a DS of at least 4.7, even more in particular a DS of at least 5.9, in the presence of the cell-impermeable molecules to be delivered across the cell membrane, wherein said crosslinked dextran nanogels and said cell-impermeable molecules are independently supplied to the cells. In another embodiment the present invention provides a method for delivery of cell-impermeable molecules across a cell membrane, said method comprising contacting cells with crosslinked dextran nanogels having a DS of at least 2.5; in particular a DS of at least 3.0; more in particular a DS of at least 3.4, more in particular a DS of at least 4.7, even more in particular a DS of at least 5.9, in the presence of the cell-impermeable molecules to be delivered across the cell membrane, wherein said crosslinked dextran nanogels and said cell-impermeable molecules are independently supplied to the cells, and wherein said cell-impermeable molecules are neutral or cationic cell-impermeable molecules with respect to the crosslinked dextran nanogels when supplied to the cell.

In another embodiment the method comprises contacting cells with crosslinked dextran nanogels having a zeta potential of at least 5 mV, in particular a zeta potential of at least 11 mV, more in particular of at least 16 mV, even more in particular of at least 21 mV in the presence of the cell-impermeable molecules to be delivered across the cell membrane. In an embodiment the present invention provides a method for delivery of cell-impermeable molecules across a cell membrane, said method comprising contacting cells with crosslinked dextran nanogels having a zeta potential of at least 5 mV, in particular a zeta potential of at least 11 mV, and more in particular of at least 16 mV, even more in particular of at least 21 mV in the presence of the cell-impermeable molecules to be delivered across the cell membrane, wherein said cell-impermeable molecules are neutral or cationic cell-impermeable molecules with respect to the crosslinked dextran nanogels when supplied to the cell. In another embodiment the method comprises contacting cells with crosslinked dextran nanogels having a DS of at least 3, in particular at least 3.4 and a cationic charge with a Zeta potential of at least 11 mV, in particular at least 16 mV, in the presence of the cell-impermeable molecules to be delivered across the cell membrane. In another embodiment the method comprises contacting cells with crosslinked dextran nanogels having a DS of at least 3, in particular at least 3.4, more in particular 4.7, even more in particular at least 5.9, and a cationic charge with a Zeta potential of at least 11 mV, in particular at least 16 mV, more in particular at least 21 mV in the presence of the cell-impermeable molecules to be delivered across the cell membrane, wherein said cell-impermeable molecules are neutral or cationic cell-impermeable molecules with respect to the crosslinked dextran nanogels when supplied to the cell. Also, in these embodiments using crosslinked dextran nanogels, the crosslinked dextran nanogel and the cell-impermeable molecules are independently supplied either simultaneous (co-delivery) or sequentially to the cells.

The methods of intracellular delivery as herein provided can be applied in any context wherein delivery of materials across the cell membrane is required, such as but not limited to drug screening, drug delivery, imaging, cell labeling, cell engineering, manufacturing of cell therapies, in particular adoptive T cell therapies, and the like. It is accordingly an object of the present invention to provide the use of the methods as herein provided in drug screening, drug delivery, imaging, cell labeling, cell engineering, manufacturing of cell therapies, adoptive T cell therapies, and the like. The methods can be applied to single cells, cell cultures, isolated cells, cells in suspension or grown on substrates such as culture dish, both in in vivo, in vitro and ex vivo applications, and typically include contacting the cells with the supramolecular polycationic materials and the cell-impermeable molecules in a solution.

In one embodiment, the supramolecular polycationic materials and the cell-impermeable molecules of the invention are independently supplied, i.e. from separate solutions, either simultaneous (co-delivery) or sequentially. In a further embodiment, the polycationic materials and the cell-impermeable molecules are part of a single composition or formulation and/or supplied as a mixture. In an even further embodiment, said mixture has been dried or lyophilized. It has been observed that cell culture media and then in particular the serum components of cell culture media may interfere with the surface charge of the supramolecular polycationic materials and accordingly affect their cell permeabilizing activity. Thus, in a preferred embodiment the cells are contacted with the supramolecular polycationic materials, in the presence of the cell-impermeable molecules to be delivered across the cell membrane, in a solution that does not compromise the cationic charge of the supramolecular polycationic material, such as a reduced serum or serum-free solution. The cells are incubated with the supramolecular polycationic materials, in the presence of the cell-impermeable molecules to be delivered across the cell membrane, for a time sufficient to achieve such delivery, such as for example from 1 minute up to 5 hours, or even more. Per reference to the examples hereinafter, already after incubation times as short as 5 minutes delivery of cell-impermeable molecules across the cell membrane can be observed. Surprisingly, incubation times up to 4 hours could be applied without being detrimental to the viability of the cells. Given the permeabilizing effect of the supramolecular polycationic materials one would have expected that such extended incubations would be toxic to the cells. This possibility of long incubation times also strongly differs from the methods of the present invention over the existing cell delivery methods, such as electroporation, that typically requires an external physical (e.g. mechanical, electrical, thermal, optical) trigger to transiently permeabilize the cell membrane over only a short period of time.

In a further aspect the present invention provides an intracellular delivery system for delivery of cell-impermeable molecules into the cell cytosol, said system comprising crosslinked cationic hydrogel nanoparticles; more in particular crosslinked dextran nanogels such as dextran methacrylate (MA)-co-TMAEMA nanogels, . . . , and the like.

In one embodiment the intracellular delivery system according to the invention comprises crosslinked cationic hydrogel nanoparticles (preferably crosslinked dextran nanogels) having a cationic charge with a zeta potential of at least 5 mV, in particular at least 11 mV, more in particular at least 16 mV, even more in particular crosslinked cationic hydrogel nanoparticles (preferably crosslinked dextran nanogels) having a cationic charge with a zeta potential of at least 21 mV.

In another embodiment the intracellular delivery system according to the invention comprises crosslinked cationic hydrogel nanoparticles (preferably crosslinked dextran nanogels) having a Degree of Methacrylate Substitution of at least 2.5, in particular at least 3.0, more in particular at least 3.4, even more in particular of at least 4.7, in a particular embodiment the crosslinked cationic hydrogel nanoparticles (preferably crosslinked dextran nanogels) have a Degree of Methacrylate Substitution of at least 5.9.

In another embodiment the intracellular delivery system according to the invention comprises crosslinked cationic hydrogel nanoparticles (preferably crosslinked dextran nanogels) having a Degree of Methacrylate Substitution of at least 3.4 and a cationic charge with a Zeta potential of at least 16 mV. In a particular embodiment the intracellular delivery system according to the invention comprises crosslinked cationic hydrogel nanoparticles (preferably crosslinked dextran nanogels) having a Degree of Methacrylate Substitution of at least 5.9 and a cationic charge with a Zeta potential of at least 21 mV.

Further to the above, the present invention could be summarized according to the following numbered embodiments.

1. A method for delivery of cell-impermeable molecules across a cell membrane, said method comprising contacting cells with supramolecular polycationic materials, in the presence of the cell-impermeable molecules to be delivered across the cell membrane, and characterized in that the cell-impermeable molecules to be delivered across the cell membrane do not interact with said supramolecular polycationic materials.

2. The method according to the first embodiment wherein the cell-impermeable molecules and supramolecular polycationic materials are independently supplied either simultaneous (co-delivery) or sequentially to the cells.

3. The method according to the previous embodiment, wherein the cell-impermeable molecules and supramolecular polycationic materials are sequentially supplied to the cells; in particular the method comprises contacting the cells with supramolecular polycationic materials and subsequently supplying the cell-impermeable molecules to be delivered across the cell membrane.

4. The method according to any one of the preceding embodiments wherein the cell-impermeable molecules are neutral or cationic cell-impermeable molecules with respect to the polycationic materials when supplied to the cell.

5. The method according to the previous embodiment wherein said neutral or cationic cell-impermeable molecules are independently supplied either simultaneous (co-delivery) or sequentially, with the membrane permeabilizing supramolecular polycationic molecules.

6. The method according to the previous embodiment said neutral or cationic cell-impermeable molecules are sequentially supplied to the cells; in particular the method comprises contacting the cells with supramolecular polycationic materials and subsequently supplying the neutral or cationic cell-impermeable molecules to be delivered across the cell membrane.

7. The method according to the previous embodiment wherein the method comprises contacting the cells with the neutral or cationic cell-impermeable molecules to be delivered across the cell membrane and subsequently supplying the supramolecular polycationic materials.

8. The method according to any one of the preceding embodiments wherein supramolecular polycationic materials are selected from supramolecular cationic nanoparticles.

9. The method according to any one of the preceding embodiments wherein the supramolecular polycationic materials are selected from crosslinked cationic polymers; in particular crosslinked cationic hydrogel nanoparticles; even more in particular crosslinked dextran nanogels such as dextran methacrylate (MA)-co-TMAEMA nanogels, and the like.

10. The method according to embodiment 9 wherein the crosslinked dextran nanogels have a Degree of Methacrylate Substitution (DS) of at least 2.5 (in particular a DS of at least 3.4, more in particular a DS of at least 5.9), or a cationic charge with a Zeta potential of at least 11 mV (in particular at least 21 mV).

11. The method according to embodiment 9 wherein the crosslinked dextran nanogels have a Degree of Methacrylate Substitution of at least 3.4 (in particular a DS of at least 4.7; more in particular a DS of at least 5.9) and a cationic charge with a Zeta potential of at least 11 mV (in particular a Zeta potential of at least 16 mV, more in particular a Zeta potential of at least 21 mV).

12. The method according to any one of the preceding embodiments, wherein the cells are contacted with the supramolecular polycationic materials and the cell-impermeable molecules in a solution.

13. The method according to embodiment 12, wherein the solution is a serum free solution.

14. The method according to any one of the preceding embodiments, wherein the cells are incubated with the cell-impermeable molecules in the presence of the supramolecular polycationic materials for at least 15 minutes, in particular for at least 15 minutes.

15. A combination or mixture of cell-impermeable molecules and supramolecular polycationic materials as provided herein, wherein the cell-impermeable molecules do not interact with said supramolecular polycationic materials, for use in medicine; in particular for use in the treatment of skin or corneal disease.

16. Use of the methods according to any one of the preceding embodiments for the in vitro or ex vivo manipulation of cells and cell lines.

17. Use of supramolecular polycationic materials, in particular of crosslinked cationic nanoparticles as defined in any one of embodiments 1 to 11, in the delivery of cell-impermeable molecules across a cell membrane and/or into a cell.

18. Use of the methods according to any one of the preceding embodiments, without the need of an external physical trigger.

19. A system for delivery of cell-impermeable molecules into the cell cytoplasm, said system comprising supramolecular polycationic materials; in particular crosslinked cationic hydrogel nanoparticles; more in particular crosslinked dextran nanogels such as dextran methacrylate (MA)-co-TMAEMA nanogels, and the like, wherein the cell-impermeable molecules are neutral or cationic cell-impermeable molecules with respect to the supramolecular polycationic materials when supplied to the cell.

20. In one embodiment the cell delivery system comprises crosslinked cationic hydrogel nanoparticles (preferably crosslinked dextran nanogels) having a cationic charge with a Zeta potential of at least 11 mV (in particular at least 16 mV, more in particular at least 21 mV), or have a Degree of Methacrylate Substitution of at least 2.5 (in particular at least 3.4 mV, more in particular at least 4.7; even more in particular at least 5.9).

21. In another embodiment the cell delivery system comprises crosslinked cationic hydrogel nanoparticles (preferably crosslinked dextran nanogels) having a Degree of Methacrylate Substitution of at least 2.5 (in particular at least 3.4 mV, more in particular at least 5.9 mV) and a cationic charge with a Zeta potential of at least 11 mV (in particular at least 16 mV, more in particular at least 21 mV).

22. The methods as herein provided or of the system as herein provided, for use as a medicine.

23. The methods as herein provided or the system as herein provided, for use in the treatment of a skin or eye/corneal disease.

24. Use of the methods as herein provided or of the system as herein provided, in the treatment of a skin or eye disease, in particular by topical or corneal administration of the supramolecular polycationic materials and the cell-impermeable molecules.

25. A method of treating a skin or eye disease, comprising administering by topical or corneal administration the supramolecular polycationic materials as defined herein and the cell-impermeable molecules to a subject.

26. The methods as herein provided or the system as herein provided, for use in adoptive T-cell therapy.

27. Use of the methods as herein provided or of the system as herein provided, in the treatment of cancer, in particular in the treatment of cancer by adoptive T-cell therapy.

28. A method of treating cancer by adoptive T-cell therapy, comprising exposing T-cells to be delivered to the patient with the supramolecular polycationic materials as defined herein and cell-impermeable molecules for use in said adoptive T-cell therapy.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIG. 1. Schematic representation of the experimental procedure and quantitative analysis to identify polycationic materials with cytosolic macromolecule delivery capacities. (a) HeLa cells were incubated for 2 h with a selected polycationic material in the presence of green fluorescent FITC-Dextran (FD10). Cell nuclei were stained with Hoechst and multiple confocal images were taken. (b) Image acquisition was performed using a 408 nm and 488 nm laser line resulting in fluorescence intensity in the blue (Hoechst) and green (FITC) channel (1 and 2). Following nuclei detection in the blue channel (2), a nuclear region of interest (ROI) was determined (3), in which the FITC signal was measured and plotted in frequency distributions for at least 200 cells per condition (4). Based on these histograms, a population of positive cells containing nuclear FD10 was determined. The relative mean fluorescence intensity (rMFI) was calculated as the nuclear MFI for a given condition divided by the nuclear MFI measured in the negative control (only FD10, without a cationic nanomaterial) and this rMFI value was used as an indicator for cytosolic FD10 delivery. MSNP: propylamine-functionalized mesoporous silica nanoparticles; DEAE: diethylaminoethyl; dextran nanogels: dextran methacrylate (MA)-co-TMAEMA nanogels; FD10: FITC-dextran 10 kDa.

FIG. 2. Dextran nanogels efficiently deliver macromolecules in HeLa cells. (a-b) Quantitative analysis of FD10 delivery in HeLa cells based on nuclear FITC fluorescence intensity, as previously described (>200 cells were imaged and analyzed per condition). Data represent the mean±SD of three independent experiments (n=3). Statistical significance relative to control (only FD10) is indicated when appropriate (ns p≥0.05, * p<0.05, ** p≤0.01, *** p≤0.001, **** p≤0.0001). Control: cells incubated with FD10 alone; FD10: FITC-dextran 10 kDa; MSNP: propylamine-functionalized mesoporous nanoparticle; DEAE: diethylaminoethyl; Dex-NG: dextran methacrylate (MA)-co-TMAEMA nanogels with a degree of substitution (DS) of 3.4; rMFI: relative mean fluorescence intensity.

FIG. 3. Impact of the methacrylate substitution degree of dextran nanogels on macromolecule delivery in HeLa cells. (a-b) HeLa cells were incubated for 2 h with dextran nanogels with a substitution degree of 3.4, 4.7 or 5.9 (dex-NG DS 3.4 (white bars), dex-NG DS 4.7 (gray bars) and dex-NG DS 5.9 (striped bars)) in the presence of FITC-labeled dextran 10 kDa (FD10). Quantitative analysis of FD10 delivery in HeLa cells based on nuclear FITC fluorescence intensity, as previously described (>200 cells were imaged and analyzed per condition). (c) Cell viability of HeLa cells as measured using an ATP-based viability assay 4 h after dextran nanogel exposure. Data represent the mean±SD of three independent experiments (n=3). Statistical significance is reported where relevant (ns p≥0.05, * p<0.05, ** p≤0.01, ** p≤0.001, **** p≤0.0001). Control: cells incubated with FD10 alone; FD10: FITC-dextran 10 kDa; dex-NG: dextran methacrylate (MA)-co-TMAEMA nanogels; DS: degree of substitution; TMAEMA: [2-(methacryloyloxy)ethyl]-trimethylammonium chloride; rMFI: relative mean fluorescence intensity.

FIG. 4. The impact of dextran nanogel cationic charge on macromolecule delivery in HeLa cells. HeLa cells were incubated for 2 h with dex-NG DS 5.9 nanogels with a zeta potential of +10 mV, +16 mV or +21 mV in the presence of FITC-dextran 10 kDa (FD10). (a-b) Quantitative analysis of FD10 delivery in HeLa cells based on nuclear FITC fluorescence intensity, as previously described (>200 cells were imaged and analyzed per condition). Data represent the mean±SD of three independent experiments (n=3). Statistical significance is reported where relevant (ns p≥0.05, * p<0.05, ** p≤0.01, p≤0.001, **** p≤0.0001). Control: cells incubated with FD10 alone; FD10: FITC-dextran 10 kDa; Dex-NG: dextran methacrylate (MA)-co-TMAEMA nanogels; DS: degree of substitution; TMAEMA: [2-(methacryloyloxy)ethyl]-trimethylammonium chloride; rMFI: relative mean fluorescence intensity.

FIG. 5. A crosslinked hydrogel network is required for nanogel-mediated macromolecule delivery in HeLa cells. HeLa cells were incubated for 2 h with freshly hydrated dex-HEMA-NG (60 μg/ml, white bars) or hydrolyzed dex-HEMA-NG (24 h pre-incubation at 37° C.) in the presence of FD10. Dex-HEMA-NG contain a hydrolysable carbonate ester in their crosslinks rendering them biodegradable in aqueous environment, in contrast to the stably crosslinked dex-NG, which were included as a control (150 μg/ml, gray bars). (a-b) Quantitative analysis of FD10 delivery in HeLa cells based on nuclear FITC fluorescence intensity, as described previously (>200 cells were imaged and analyzed per condition). Data represent the mean±SD of three independent experiments (n=3). Statistical significance is reported where relevant (ns p≥0.05, * p<0.05, ** p≤0.01, p≤0.001, **** p≤0.0001). Control: cells incubated with FD10 alone; FD10: FITC-dextran 10 kDa; rMFI: relative mean fluorescence intensity; dex-HEMA-NG: dextran-hydroxyethylmethacrylate (dex-HEMA)-co-TMAEMA nanogels; dex-NG: dextran-methacrylate (MA)-co-TMAEMA nanogels.

FIG. 6. Dextran nanogels can effectively deliver FITC dextrans of up to 40 kDa in HeLa cells. HeLa cells were incubated for 2 h with dex-NG DS 5.9 nanogels (gray bars) in the presence of FITC-dextran with an average size of respectively 4, 10, 20 or 40 kDa (FD4, FD10, FD20 or FD40). (a-b) Quantitative analysis of FD4-40 delivery in HeLa cells based on nuclear FITC fluorescence intensity, as described previously (>200 cells were imaged and analyzed per condition). Data represent the mean±SD of three independent experiments (n=3). Statistical significance is reported where relevant (ns p≥0.05, * p<0.05, ** p≤0.01, *** p≤0.001, **** p≤0.0001). Control: cells incubated with FD4-40 alone (white bars); dex-NG: dextran methacrylate (dex-MA)-co-TMAEMA nanogels; rMFI: relative mean fluorescence intensity.

FIG. 7. Dextran nanogel-mediated cytosolic FITC dextran delivery is endocytosis-independent. HeLa cells were incubated for 2 h with dex-NG DS 5.9 nanogels, loaded with Cy5-labeled RNA (Cy5-dex-NG), in the presence of FITC-dextran 10 kDa (FD10). (a) Scatter plots between FD10 delivery (nuclear rMFI FITC) and Cy5-dex-NG uptake was investigated using simple linear regression analysis for 86 cells. The dashed line represents the 95% confidence band of the regression line. The magnitude (between 0 and 1) of the regression coefficient indicates the strength of correlation (R²=0.015). A p value below 0.05 indicates that the slope of the curve significantly differs from 0 (p=0.26). (b) Correlation between FD10 delivery (rMFI FITC) and total amount of Cy5-dex-NG containing endosomes in individual cells was investigated using simple linear regression analysis (86 cells, R²=0.002, p=0.67). Experimental data shown are one representative of three independent experiments with a minimum of 50 cells analyzed per repeat. Control: cells incubated with FD10 alone; rMFI: relative mean fluorescence intensity; dex-NG: dextran methacrylate (dex-MA)-co-TMAEMA nanogels.

FIG. 8. Dextran nanogel (HyPore)-mediated FD10 delivery outperforms nucleofection in primary human T cells. Human T cells were isolated from peripheral blood mononuclear cells and expanded for several weeks. Next, cells were resuspended in Opti-MEM (gray bars) or Opti-MEM supplemented with 10 mM N-acetylcysteine (NAC) (striped bars) and incubated for 1 h with dextran nanogels (12, 25 or 50 μg/ml) in the presence of FITC-dextran 10 kDa (FD10). As a control, cells were treated with nucleofection (program E0-115, Lonza Nucleofector, white bars) (a) Quantitative analysis of FD10 delivery efficiency using flow cytometry. Cells were stained with a membrane exclusion dye (TO-PRO-3 iodide) staining dead cells. TO-PRO-3 iodide negative cells were gated for delivery efficiency analysis. (b) Analysis of cell viability, 4 h after treatment, using an ATP-based viability assay (CellTiter-Glo®). (c-d) Quantitative analysis of FD10 delivery efficiency using flow cytometry on TO-PRO-3 iodide negative cells. The yield is determined as percentage living cells (based on CellTiter-Glo® assay), multiplied by the percentage of FD10-positive cells. Data shown as the mean±SD of three independent experiments (n=3). Statistical significance is reported where relevant (ns p≥0.05, * p<0.05, ** p≤0.01, *** p≤0.001, **** p≤0.0001). Control: cells incubated with FD10 alone; dextran nanogels: dextran methacrylate (MA)-co-TMAEMA nanogels; rMFI: relative mean fluorescence intensity. (e) Flow cytometry histograms displaying FD10 delivery efficiency upon nucleofection or treatment with 25 μg/ml of dextran nanogels in the presence or absence of 10 mM NAC.

FIG. 9. HyPore-mediated delivery of functional cargo. HeLa cells were incubated for 2 h with HyPore (i.e. dex-MA NGs DS 5.9) in the presence of Histone-Label ATTO488 (nanobody), after which the cells were washed and the nuclei stained with Hoechst (blue). (a-b) Quantitative analysis of Histone-label delivery in HeLa cells based on nuclear ATTO488 fluorescence intensity. Control: incubated with Histone-label alone; HL: Histone-Label ATTO488 (nanobody). (c) HeLa reporter cells containing the reporter plasmid, pLV-CMV-LoxP-DsRed-LoxP-eGFP, were incubated for 2 h with HyPore in the presence of Cre recombinase. Upon successful delivery of active Cre recombinase, the DsRed gene is floxed and the eGFP gene is expressed resulting in green fluorescent signal. The graph shows the quantitative analysis of functional Cre recombinase delivery in HeLa reporter cells, analyzed using flow cytometry. HeLa reporter cells were incubated for 2 h in the presence of Cre recombinase and HyPore. Both Nucleofection and PULSin (complexed with Cre recombinase) treatment were performed according to the manufacturers' instructions. Data represent the mean±SD of three biological replicates (n=3). Statistical significance is reported where relevant (ns p≥0.05, * p<0.05, ** p≤0.01, *** p≤0.001, **** p≤0.0001). Control: incubated with Cre recombinase alone; Cre: Cre recombinase enzyme; HyPore: Dextran methacrylate (MA)-co-TMAEMA nanogels. (d) HyPore mediated delivery of gadobutrol in human T cells for magnetic resonance imaging (MRI). Human T cells were incubated for 1 h in Opti-MEM containing HyPore and gadobutrol (Gd-DO3A-butrol). Gadubutrol is an MRI contrast agent that provides a strong T1 brightening signal. As a positive control, gadobutrol was delivered in human T cells using nucleofection. Normalized relaxation values (R₁=1/T₁) are shown. Data represent the mean±SD of at least two biological replicates (n=2). Gd: gadolinium; HyPore: Hydrogel-enabled nanoPoration via dextran methacrylate (dex-MA)-co-TMAEMA nanogels; NTC: non-treated condition; Control: incubated with gadobutrol alone.

FIG. 10. Dex-HEMA nanogels degrade over time while dex-MA nanogels remain stable in an aqueous environment. The degradation kinetics of dex-NG and dex-HEMA-NG in HEPES buffer pH 7.4, 20 mM at 37° C. was measured by dynamic light scattering. Data were normalized to 1 for comparison. Scattering intensity, measured with a 5 min interval, is plotted as a function of time. Dex-NG: dextran methacrylate (MA)-co-TMAEMA nanogels; dextran hydroxyethylmethacrylate (HEMA)-co-TMAEMA nanogels.

FIG. 11. Dextran nanogels rapidly deliver propidium iodide into HeLa cells. HeLa cells were incubated for 1 h in the presence of dex-NG and propidium iodide (PI). Quantitative analysis of PI delivery in HeLa cells using confocal microscopy images. Data represent the mean±SD of three biological replicates (n=3). Dex-NG: dextran methacrylate (MA)-co-TMAEMA nanogels; rMFI: relative mean fluorescence intensity.

FIG. 12. Increased delivery in NAC-treated T cells is not ROS-mediated and cell type specific. (a) 2-mercaptoethanol (2ME)-treated human T cells do not show improved delivery efficiency when treated with dex-NG. Human T cells were resuspended in Opti-MEM with (gray bars) or without (white bars) the ROS-scavenger 2ME and incubated for 1 h with dex-NG in the presence of FD10. Quantitative analysis of FD10 delivery efficiency in human T cells using flow cytometry and analysis of T cell viability, 4 h after treatment using an ATP-based viability assay. (b) NAC-treated HeLa cells do not show improved FD10 delivery efficiency when treated with dex-NG. HeLa cells were incubated with dex-NG and FD10 in Opti-MEM containing the ROS-scavenger N-Acetylcysteine (NAC). Quantitative analysis of FD10 delivery in HeLa cells based on nuclear FITC fluorescence intensity using confocal microscopy images. Data represent the mean±SD of two biological replicates (n=2). Statistical significance is reported where relevant (ns p≥0.05, * p<0.05, ** p≤0.01, **** p≤0.001**** p≤0.0001). Control: incubated with FD10 alone; FD10: FITC-dextran 10 kDa; dex-NG: dextran methacrylate (MA)-co-TMAEMA nanogels; rMFI: relative mean fluorescence intensity.

FIG. 13. Dextran nanogel-treated cells quickly regain membrane integrity after treatment. Human T cells quickly regain membrane integrity after dex-NG treatment. Human T cells were treated for 1 h with dex-NG and FD10 in Opti-MEM containing N-acetyl cysteine (NAC). After treatment, T cells were washed and stained with TO-PRO-3 iodide. (a) Contour density plots show delivery of FD10 versus TO-PRO-3 iodide signal. (b) The majority of T cells are not stained by TO-PRO-3, indicating an intact cell membrane. Data represent the mean±SD of two biological replicates (n=2). Statistical significance is reported where relevant (ns p≥0.05, * p<0.05, ** p≤0.01, *** p≤0.001, **** p≤0.0001). FD10: FITC dextran 10 kDa; Dex-NG: dextran methacrylate (MA)-co-TMAEMA nanogels with DS 5.9; PBCEC: primary bovine corneal epithelial cells; Control: incubated with FD10 alone; rMFI: relative mean fluorescence intensity; TO-PRO-3: TO-PRO-3 iodide, a membrane-impermeable staining dye.

FIG. 14. Dextran nanogels efficiently deliver FD10 in the murine, macrophage-like cell line RAW 264.7. RAW 264.7 cells were incubated for 2 h with dex-NG in the presence of FD10. (a-b) Quantitative analysis of FD10 delivery in RAW 264.7 using confocal microscopy images. Data represent one biological sample (n=1). Control: incubated with FD10 alone; FD10: FITC-dextran 10 kDa; Dex-NG: dextran methacrylate (MA)-co-TMAEMA nanogels; rMFI: relative mean fluorescence intensity.

FIG. 15. Dextran nanogels efficiently deliver FD10 in primary bovine corneal epithelial cells. Primary bovine corneal epithelial cells (PBCEC) were incubated for 2 h with dex-NG in the presence of FD10. (a-b) Quantitative analysis of FD10 delivery in PBCEC using confocal microscopy images. (c) Viability in PBCEC was measured using an ATP-based viability assay. Data represent the mean±SD of three biological replicates (n=3). Statistical significance is reported where relevant (ns p≥0.05, * p<0.05, ** p≤0.01, *** p≤0.001, **** p≤0.0001). Control: incubated with FD10 alone; FD10: FITC-dextran 10 kDa; Dex-NG: dextran methacrylate (MA)-co-TMAEMA nanogels; rMFI: relative mean fluorescence intensity.

FIG. 16. Hydrogel-enabled nanoPoration (HyPore) mediated delivery of granzyme A in HeLa cells inducing apoptosis. HeLa cells were incubated for 2 h with HyPore (dex-NGs DS 5.9) in the presence of granzyme A (GrzA). Quantitative analysis of functional GrzA delivery was performed using an ATP-based viability assay 24 h after treatment. Data represent the mean±SD of three biological replicates (n=3). Statistical significance is reported where relevant (ns p≥0.05, * p<0.05, ** p≤0.01, *** p≤0.001, **** p≤0.0001). Control: incubated with granzyme A alone; HyPore: incubated with HyPore nanogels alone; GrzA: Granzyme A.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous. When describing the compounds of the invention, the terms used are to be construed in accordance with the following definitions, unless a context dictates otherwise.

The term “about” or “approximately” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

As used in the specification and the appended claims, the singular forms “a”, “an”, and “the” include plural referents unless the context clearly dictates otherwise. By way of example, “an extract” means one extract or more than one extract.

The term “supramolecular polycationic materials” as used herein, and hereinafter also referred to as “polycationic materials”, generally refers to chemical systems comprising polymeric materials that are spatially organized by intermolecular forces, including weak intermolecular forces, like electrostatic charge, or hydrogen bonding to strong covalent bonding; and bearing positive charges. In a particular embodiment, the polymeric materials present within the supramolecular polycationic materials, are cationic polymeric systems typically synthesized in the presence of novel cationic entities, and incorporating said cationic entities on their backbone and/or as side chains. Examples of supramolecular polycationic systems include, but are not limited to polycationic scaffolds, porous networks, hydrogels, fibers, colloidal materials or other assemblies. More in particular, such systems include crosslinked cationic polymers, cationic polymer nanoparticles and cationic nanogels. Examples of suitable polycationic materials in said supramolecular systems include, but are not limited to natural or semi-synthetic cationic polymers (e.g. chitosan, cationic dextran, cationic cellulose, cationic gelatin, cationic cyclodextrin, poly(L-lysine), poly(L-arginine), poly(L-histidine), polymers containing natural oligoamines such as spermine, spermidine, putrescine), synthetic cationic polymers (poly(ethylene imine), PAMAM dendrimers, DEAE-dextran, poly(2-(dimethylamino) ethyl methacrylate, poly(β-amino esters) and other amine-containing polyesters, poly(amido amines), poly(N,N-dimethyldiallylammonium) chloride, poly(N-alkyl-4-vinylpyridinium) bromide or other quaternary ammonium containing polymers, polyolefins with cationic side groups, polyhexamethylene biguanide and its derivatives) and cationic nanoparticles (e.g. cationized inorganic nanoparticles such as modified gold nanoparticles, silver nanoparticles, iron oxide nanoparticles, silica nanoparticles, including propylamine functionalized mesoporous silica nanoparticles; cationized organic nanoparticles such as carbon-based nanoparticles, poly(dopamine) nanoparticles, polystyrene nanoparticles or modifications and combinations thereof. The supramolecular polycationic systems can be responsive to external stimuli (e.g. hydrolysable, responsive to pH, temperature, enzymes, ionic strength, light, magnetic field, electric field, redox and chemicals). As used herein “cationization” refers to the modification of materials with positively charged sites, e.g. through chemical reaction with cationic reactive agents, coating with cationic materials such as cationic polymers, application of cationic surfactants etc. As evident from the examples hereinafter, crosslinked cationic polymers and crosslinked cationic nanoparticles; more in particular crosslinked cationic hydrogel nanoparticles; even more in particular crosslinked dextran nanogels such as dextran methacrylate (MA)-co-TMAEMA nanogels, dextran hydroxyethyl methacrylate (HEMA)-co-TMAEMA, and the like, in contrast to soluble cationic polymers, can effectively permeabilize the plasma membrane of different cell types without excessive cell damage, allowing highly efficient cytosolic delivery of several classes of molecules.

The term “cell-impermeable molecules” as used herein generally refers to any molecule often also referred to as “cargo” molecule, incapable of passively crossing the cell membrane of a cell. It typically includes macromolecular hydrophilic cargo such as RNAs, DNAs, proteins, glycoproteins, peptides, ribonucleoproteins, i.e. cargo normally delivered across a cell membrane by a nanocarrier (e.g. a lipid or polymer-based nanocarrier) or a physical stimulus like electroporation, across a cell membrane. In the context of the invention, cell delivery in the presence of the aforementioned polycationic materials was enhanced in the absence of an interaction between the cargo and the polycationic materials. This in contrast to the typical co-delivery wherein the supramolecular polycationic materials act as carriers for the cargo, the cargo being contained inside the carrier or on the surface of the nanocarrier and released from the carrier inside the cell following endocytosis and endosomal escape. The aforementioned supramolecular polycationic materials related to the invention have a different behavior. Instead of carrying the cargo, they act to permeabilize the cell membrane and allow the cargo molecules to cross the membrane. As such the presence of the supramolecular polycationic materials related to the invention enable a passive transport of the cell-impermeable molecules (the cargo) across the cell-membrane. The transport is based on the absence of an electrostatic attraction between the cargo and the supramolecular polycationic materials.

This explains why the best results are obtained in case the cell-impermeable molecules do not interact with said supramolecular polycationic material, in particular in case the cell-impermeable molecules are neutral or positive charged with respect to the supramolecular polymers. Thus in a particular embodiment the cargo molecules are neutral or cationic cell-impermeable molecules with respect to the polycationic materials when supplied to the cell.

Expressed differently, in the environment wherein the polycationic materials and cargo molecules are supplied to the cell, the former are positive of charge and the latter are either free of charge (neutral or zwitterionic (equal number of positive and negative charge)) or positive of charge (cationic). In a preferred embodiment the supramolecular polycationic materials and the cell-impermeable molecules are each independently applied to the cell medium, and both free in solution. It will be clear to the skilled artisan that the charge of the cargo molecules can be influenced, amongst others, by the pH or ionic strength of the medium wherein the cell is incubated with the cargo and the polycationic materials. In one embodiment, the invention has been shown especially efficient for delivery of large cargo molecules, hence molecules of e.g. up to 200 kDa in size can be delivered. More specific, cargo molecules of up to 100 kDa in size can be delivered, and in a particular embodiment delivery of cargo molecules of up to 80 kDa, more in particular of up to 75, 70, 65, 60, 55, 50 or 45 kDa in size, and even more particular of up to 40 kDa in size is provided. Any type of cargo can be delivered but specifically envisaged are peptides, proteins, including functional proteins such as nanobodies and enzymes, and imaging or contrast agents. In a further embodiment, neutral or neutralized nucleic acids or nucleic acid derivatives (e.g. phosphotriester RNA or DNA) can be delivered.

A “serum free solution” or “serum free medium” as used herein generally refers to cell culture media that does not contain a nutrient and growth factor-rich serum derived from animal or human blood. Serum-free media uses synthetic or purified ingredients to provide nutrients and growth factors that support growth and survival of cells in culture. Serum is the amber fluid rich in protein that is separated from coagulated blood. Serums like newborn or fetal bovine serums are commonly used in cell culture media to provide nutrients and growth factors that promote survival and growth of cells. In a serum free solution synthetic or purified ingredients to provide nutrients and growth factors that support growth and survival of cells in culture, are used instead.

As already mentioned hereinbefore, best results were obtained with polycationic materials with a sufficiently high cationic charge (i.e. zeta potential of at least +11 mV, in particular at least +16 mV; more in particular at least +21 mV) could successfully deliver cargo across the membrane. In practice, the zeta potential of a dispersion of polycationic materials is measured by applying an electric field across the dispersion. Particles within the dispersion with a zeta potential will migrate toward the electrode of opposite charge with a velocity proportional to the magnitude of the zeta potential. This velocity is measured using the technique of the laser Doppler anemometer. The frequency shift or phase shift of an incident laser beam caused by these moving particles is measured as the particle electrophoretic mobility, and this mobility is converted to the zeta potential by inputting the dispersant viscosity and dielectric permittivity, and the application of the Smoluchowski theories (see for example Zeta Potential Using Laser Doppler Electrophoresis—Malvern.com). In the examples, the Zeta potential was acquired in HEPES buffer (20 mM, pH 7.4) using a Zetasizer Nano ZS (Malvern), equipped with Dispersion Technology Software.

Aside from use in vivo, the method of the invention provides a highly versatile and cost-effective technique for high-throughput ex vivo manipulation of primary cells and cell lines. A variety of cell types can be transfected, including hard-to-transfect primary corneal epithelial cells and primary human T cells. The present invention equally provides the present finding in an in vitro method for delivery of cell-impermeable molecules across the cell membrane. In either of said applications the cells are incubated with said molecules in the presence of the supramolecular polycationic materials. There is no particular limitation in the presentation of the cells to the cell-impermeable molecules and the supramolecular polycationic materials. The cells can be free in suspension or are adhered to for example a multi-well plate. Also on the actual exposure of the cells to both the cell-impermeable molecules and the supramolecular polycationic materials, no particular order was found to influence the delivery of the cargo across the membrane. Again, also in this in vitro method, the cell-impermeable molecules and the supramolecular polycationic materials are independently supplied either simultaneous (co-delivery) or sequentially to the cells.

As mentioned herein before, the methods of the present invention are particularly suitable for the delivery of cell-impermeable molecules to the skin epithelium, and more particularly for topical skin applications for treatment of skin disorders and maladies. Skin maladies and disorders range from temporary dry skin caused by environmental conditions to serious illnesses which can cause incapacitation and death. Included in this range are dry skin, severe dry skin, dermatitis, psoriasis, eczema, terosis, dandruff, ichthyosis, keratoses, pruritis, age spots, cradle cap, lentigines, scales, melasmas, wrinkles, stretch marks, dermatoses, minor burns and erythema. It is an object of the present invention to provide the use of supramolecular polycationic materials and the methods as herein described in the treatment of skin disorders and maladies; in particular selected from dryskin, severe dryskin, dermatitis, psoriasis, eczema, terosis, dandruff, ichthyosis, keratoses, pruritis, age spots, cradle cap, lentigines, scales, melasmas, wrinkles, stretch marks, dermatoses, minor burns and erythema. In such application the cell-impermeable molecules will include therapeutic, dermatological, pharmaceutical, medical, and/or cosmetic compositions such as those that improve or eradicate itching, irritation, pain, inflammation, age spots, keratoses, wrinkles, and other blemishes or lesions of the skin. By way of example and not by way of limitation: analgesics, anesthetics, antiacne agents, antibacterial agents, anti-yeast agents, anti-fungal agents, antiviral agents, antibiotic agents, porbiotic agents, anti-protozal agents, anti-pruritic agents, antidandruff agents, anti-dermatitis agents, anti-emetics, anti-inflammatory agents, anti-hyperkeratolyic agents, anti-dry skin agents, antiperspirants, anti-psoriatic agents, anti-seborrheic agents, hair conditioners, hair treatments, hair growth agents, anti-aging agents, anti-wrinkle agents, antihistamine agents, disinfectants, skin lightning agents, depigmenting agents, vitamins and vitamin derivatives, gamma-linolenic acid (GLA), beta carotene, quercetin, asapalene, melaluca alternifolia, dimethicone, neomycin, corticosteroids, tanning agents, zinc/zinc oxides, sulfur agents, hormones, retinoids, clotrimazole, ketoconazole, miconazole, griseofulvin, hydroxyzine, diphenhydramine, pramoxine, lidocaine, procaine, mepivacaine, monobenzone, erythidocaine, erythromycin, tetracycline, clindamycin, meclocline, hydroquinone, minocycline, naproxen, ibuprofen, theophylline, cromolyn, alburterol, retinoic acid and its derivatives, hydrocortisone and its derivatives, mornetasone, desonide, trimcinolone, predisolone, NUTRACORT® brand topical steroid application, salicylic acid, phospholipids, calamine, allantoin, isohexadelane, ceresin, galcipotriene, DOVONEX® brand dermatological preparation, anthralin, betamethasone valerate, betamethasone diproprionate, trimcinolone acetonide, fluocinonide, clobetasol propionate, benzoyl peroxide, crotamition, propranolon, promethanzine, vitamin A palmitate, vitamin E acetate, vitamin D and mixtures or derivatives thereof.

Besides topical applications to skin, the methods of the present invention are equally useful in the delivery of cell-impermeable molecules to the cornea. Consequently in a further embodiment the present invention provides the methods and/or systems according to the invention for use in in the treatment of corneal diseases, particularly disorders in the anterior epithelium of cornea. The corneal disease in the present invention indicates conditions of injured cornea caused by various factors, specifically including keratitis caused by physical/chemical irritation, allergy, bacteria/fungi/virus infections, etc., as well as corneal ulcer, abrasion of the anterior epithelium of cornea (corneal erosion), edema of the anterior epithelium of cornea, corneal burn, corneal corrosion by chemicals, dry-eye, and the like. When used in the treatment of corneal disorders, the cell-impermeable molecules, will include therapeutic ingredients for a corneal disease, for example, hyaluronic acid or its salt, chondroitin sulfate or its salt, the enzyme hyaluronidase other enzymes, anesthetics, vitamins, zinc, antibiotics, anti-allergic agents, carbamide, cytokinases, vasoconstrictors, anti-viral agents, anti-fungal agents, anti-inflammatory agents, lubricants and the like. In a preferred embodiment of the corneal application, the cell-impermeable molecules and the supramolecular polycationic materials are provided as an ophthalmic solution, optionally comprising as further ingredients buffer, tonicity agent, solubilizer, surfactant, stabilizer, preservative, pH adjuster, and the like.

The optional ingredients will depend on the application and will be determined by the skilled person, are exemplified specifically by a buffer such as potassium dihydrogen phosphate, sodium hydrogen phosphate, boric acid, sodium borate, sodium citrate, sodium acetate, monoethanolamine, trometamol, and the like; a tonicity agent such as sodium chloride, potassium chloride, glycerin, glucose, and the like; a solubilizer such as ethanol, castor oil, and the like; surfactant such as polysorbate 80, polyoxyethylene hardened castor oil, and the like; a stabilizer such as sodium ethylenediaminetetraacetate and the like; a preservative such as benzalkonium chloride, benzethonium chloride, chlorobutanol, benzyl alcohol, and the like, and a pH adjuster such as hydrochloric acid, sodium hydroxide, and the like.

Recent advances in diagnostic imaging, such as magnetic resonance imaging (MRI), computerized tomography (CT), single photon emission computerized tomography (SPECT), and positron emission tomography (PET) have made a significant impact in cardiology, neurology, oncology, and radiology. Although these diagnostic methods employ different techniques and yield different types of anatomic and functional information, this information is often complementary in the diagnostic process. The methodology requires the localized delivery of imaging agents to the body. Having identified a novel approach of delivering cell-impermeable molecules to the cell, the methods of the present invention can provide an alternative in the delivery of cell-impermeable imaging agents with diagnostic imaging applications. According to a particular embodiment of the present invention, the systems and methods of the present invention are used for imaging, especially medical imaging.

An unexpected finding is the strongly improved cytosolic delivery in primary human T cells when using the clinically approved N-acetyl cysteine (NAC) in the methods of the invention. NAC is clinically approved for various medical uses (e.g. paracetamol overdose, chronic obstructive pulmonary disease) and has recently shown to markedly increase the efficacy of adoptive T cell therapy by improving both T cell mediated tumor control and survival in mice. Indeed, ex vivo treatment with NAC during T cell activation and expansion has demonstrated improved differentiation into stem cell memory T cells, a T cell phenotype that enables superior in vivo persistence. Given the above, the methods of the present invention, in particular the combination of a supramolecular polycationic material with NAC, more in particular the combination of cationic dextran nanogels with NAC, can likewise be applied for the cytosolic delivery of membrane-impermeable cargo to T cells in the context of adoptive T cell therapy.

The following examples are set forth below to illustrate the methods, compositions, and results according to the disclosed subject matter. These examples are intended to illustrate representative methods, compositions, and results. These examples are not intended to exclude equivalents and variations of the present invention, which are apparent to one skilled in the art.

### EXAMPLES

### Materials and Methods

### Materials

FITC-labeled dextrans (4 kDa, 10 kDa, 20 kDa and 40 kDa), N-acetyl cysteine (NAC), dispase II, dextran sulfate sodium salt (10 kDa), DEAE-dextran (20 kDa), propylamine functionalized mesoporous silica nanoparticles and sorbitol-supplemented hormonal epithelial medium were obtained from Sigma-Aldrich (Overijse, Belgium). Hoechst 33342 was purchased from Molecular Probes™ (Belgium). CellTiter-Glo® was obtained from Promega (Leiden, Netherlands). TO-PRO™-3 iodide, penicillin, Annexin V (FITC conjugate), DMEM/F12, DMEM, IMDM, RPMI, phosphate buffered saline (PBS), CO₂-independent medium and 1% agar were acquired from Invitrogen (Merelbeke, Belgium). Histone-Label ATTO488 was obtained from Chromotek (Planeg-Martinsried, Germany). Cre recombinase was purchased from New England Biolabs (Mississauga, Canada). Human recombinant granzyme A was purchased from Biolegend (San diego, USA). Gadavist® (gadobutrol) was acquired from Bayer (Leverkusen, Germany). Puromycin was purchased from Gibco (Camarillo, USA). Lymphoprep was purchased from Alere Technologies AS (Oslo, Norway). Immunocult Human CD3/CD28 T cell Activator was from Stemcell Technologies (Vancouver, Canada). Fetal bovine serum was purchased from Hyclone (GE Healthcare, Machelen, Belgium). Bovogen (Melbourne, Australia) provided the Fetal calf serum (FCS). CELLview™ culture dishes were purchased from Greiner Bio-One GmbH (Vilvoorde, Belgium). Phytohemagglutinin was purchased from Remel Europe (KENT, UK). IL-2 was purchased from Roche Diagnostics (Mannheim, Germany). PULSin and JetPEI® were obtained from Polyplus Transfection (Strasbourg, France). Fluorescent CTRL siRNA labeled with a Cy5 dye at the 5′ end of the (sense) strand (abbreviated Cy5-RNA) was provided by Eurogentec (Seraing, Belgium).

### Nanoparticle Synthesis, Preparation and Characterization

Dextran methacrylate (MA)-co-TMAEMA nanogels (dex-NG) and dextran hydroxyethyl methacrylate (HEMA)-co-TMAEMA nanogels (dex-HEMA-NG) were synthesized by photopolymerizing respectively dextran methacrylate (dex-MA) or dextran hydroxyethyl-methacrylate (dex-HEMA), with the indicated substitution degrees, with the cationic methacrylate monomer [2-(methacryloyloxy)ethyl]-trimethylammonium chloride (TMAEMA), using an inverse emulsion method as previously described⁵⁰. Following their synthesis, the nanogels were lyophilized and stored dessicated. To obtain nanogels for in vitro experiments, a weighted amount of lyophilized nanogels was dispersed in RNase free water followed by sonication (3×5 s amplitude 10%) using a Branson Digital Sonifier® (Danbury, USA). Propylamine-functionalized mesoporous silica nanoparticles were likewise dispersed in RNase free water before experimental use and sonicated (3×2 min, amplitude 15%, 10 sec on/10 sec off). Zeta-potential and hydrodynamic diameter of NGs and MSNPs were acquired in HEPES buffer (20 mM, pH 7.4) using a Zetasizer Nano ZS (Malvern), equipped with Dispersion Technology Software.

### Cell Lines, Primary Cells and Cell Culture Conditions

HeLa cells were obtained from American Type Culture Collection (ATCC, Manassas, USA) and cultured in DMEM/F12 supplemented with 10% heat-inactivated FBS, 2 mg/ml L-glutamine and 100 U/ml penicillin/streptomycin. HeLa cells containing the Cre reporter construct pLV-CMV-LoxP-DsRed-LoxP-eGFP-IRES-Puro were kindly provided by Dr. O.G. de Jong and Dr. P. Vader (University Medical Center Utrecht) (21). These cells were cultured in DMEM supplemented with 10% heat-inactivated fetal bovine serum, 2 mg/ml L-glutamine, 100 U/ml penicillin/streptomycin and 2 μg/ml puromycin.

Buffy coats were obtained with informed consent from healthy donors and used following the guidelines of the Medical Committee of the Ghent University Hospital (Belgium). Peripheral blood mononuclear cells (PBMCs) were isolated from buffy coats via density centrifugation using Lymphoprep. Next, PBMCs were stimulated with Immunocult Human CD3/CD28 T cell Activator and cultured in IMDM supplemented with 10% fetal calf serum, 100 U/ml penicillin, 100 μg/ml streptomycin, 2 mM glutamine and 5 ng/ml IL-2 for 7 days. Subsequently, the PBMCs were harvested and maintained in complete IMDM supplemented with 5 ng/ml IL-2. When required, T cells were restimulated using a PBMCs and JY feeder cell mixture and 1 μg/ml phytohemagglutinin. Feeder cells were irradiated using the Small Animal Radiation Research Platform (Xstrahl, Surrey, UK) at respectively 40 Gy and 50 Gy before use. Resting CD3+ cells (referred to as human T cells) were harvested 14 days after stimulation and used for experiments as further indicated.

Freshly excised bovine eyes were collected at a local slaughterhouse (Flanders Meat Group, Zele, Belgium) in cold CO₂-independent medium. Within 30 min following collection, excess tissue was removed and the eyes were disinfected by dipping into a 5% ethanol solution. A trephine blade was used to collect 10 mm diameter corneal buttons. The corneal buttons were rinsed with DMEM containing antibiotics and divided in 4 equal parts using a scalpel, rinsed again with DMEM and placed in a 15 mg/ml Dispase II, 100 mM SHEM solution at 37° C. for 10 min. Hereafter the tissues were rinsed with PBS and placed in a fresh Dispase II-containing medium and kept at 4° C. overnight. The following day the epithelial layer was separated from the corneal stroma using a blunt stainless steel spatula. To obtain a single cell solution the epithelial cells were placed in 1 ml of preheated (37° C.) 0.25% trypsin/1 mM EDTA and incubated for 5 min at 37° C. To neutralize the trypsin, cell medium containing FBS was added after incubation. The cells were collected via centrifugation (2 min, 1000 rpm) and resuspended in fresh SHEM medium and cultured as described earlier.

### Cationic Nanocarrier-Induced Cytosolic Delivery in HeLa Cells

HeLa cells were seeded at 50.000 cells per compartment in a 4 compartment, 35 mm diameter glass bottom CELLview™ culture dish (Greiner Bio-One GmbH, Vilvoorde, Belgium). After 24 h, cells were washed twice using PBS. Next, cells were incubated in Opti-MEM containing the indicated nanomaterial and either FITC-dextran, Histone-Label ATTO488, Cre recombinase or granzyme A at the specified concentrations. Incubations were performed for 2 h at 37° C. in a humidified atmosphere containing 5% CO₂ unless specified otherwise. Next, nanocarriers and excess proteins were washed away using PBS. Cell nuclei were stained in cell culture medium containing 20 μg/ml Hoechst 33342 for 15 min. Finally, staining solution was removed and fresh cell culture medium was added. Cells were kept at 37° C. in humidified atmosphere with 5% CO₂ until confocal imaging.

### Quantification of FITC-Dextran and Histone-Label ATTO488 Delivery in HeLa Cells and Visualization of Human T Cells

Hoechst-stained HeLa cells were imaged using a spinning disk confocal (SDC) microscope, equipped with a Yokogawa CSU-X confocal spinning disk device (Andor, Belfast, UK), a MLC 400 B laser box (Agilent technologies, California, USA) and an iXon ultra EMCCD camera (Andor Technology, Belfast, UK). A Plan Apo VC 60×1.4 NA oil immersion objective lens (Nikon, Japan) was used for imaging adherent cell types while human T cells were imaged using a Plan Apo VC 60×1.2 NA water immersion lens (Nikon, Japan). NIS Elements software (Nikon, Japan) was applied for imaging. Hoechst 33342 staining and FITC-dextran or Histone-Label ATTO488 were excited sequentially with 0.2 s delay using a 405 nm (Hoechst 33342) and 488 nm (FITC-dextran or Histone Label ATTO488) laser line. ImageJ (FIJI, Version 1.8.0) software was used to analyze cellular delivery. Nuclei were detected in the blue channel using thresholding, excluding nuclei at the image border. The same threshold settings were maintained for every image. The indicated nuclear region of interest (ROI) was then applied to the green channel to determine the nuclear green fluorescence. A minimum of 200 cells was analyzed per condition unless specified otherwise. These intensity values were plotted as frequency distributions (histograms) and used to determine the percentage of positive cells containing FITC-dextran or Histone-Label ATTO488. The relative MFI was determined as the average mean gray values measured in the green channel (as previously described) divided by the average mean gray value measured in the negative control (i.e. cargo only).

### Quantification of Cell Viability

The toxicity of cationic nanomaterials on HeLa cells, PBCEC cells (2×10⁴ cells per well) and human T cells (1×10⁶ cells per well) was measured using a CellTiter-Glo® luminescent viability assay (Promega, Belgium) according to the manufacturer's instructions. Cells were seeded 24 h before treatment in a 96-well plate and treated as previously described, incubating them for 2 h (1 h for human T cells) in the presence of a cationic nanomaterial and a cargo molecule. Next, cells were washed and new cell culture medium was added. After 4 h, medium was renewed and an equal volume of CellTiter-Glo® reagent was added. Samples were shaken on a shaker plate for 10 min at 100 rpm. One hundred μl solution was taken from each sample and transferred to an opaque 96-well plate (Greiner Bio-One GmbH, Vilvoorde, Belgium). Sample luminescence was measured using a microplate reader (GloMax®).

### Correlating Endosomal Uptake with Cytosolic Delivery in HeLa Cells

HeLa cells were seeded at 5×10⁴ cells per compartment in a 4 compartment, 35 mm diameter glass bottom CELLview™ culture dish. After 24 h, the cells were washed twice with PBS. Cationic dextran nanogels were fluorescently labeled by mixing them for 15 min with Cy5-RNA to allow electrostatic complexation. Next, HeLa cells were incubated for 2 h in Opti-MEM containing Cy5-RNA loaded dextran nanogels (Cy5-dex-NG) and 2 mg/ml FITC-dextran 10 kDa (FD10). Excess Cy5-dex-NG and FD10 were washed away using PBS, followed by a short washing step with 1 mg/ml dextran sulfate sodium salt (10 kDa, Sigma-Aldrich) in PBS. Finally, cells were washed using PBS and incubated in cell culture medium containing 20 μg/ml Hoechst 33342. Staining solution was removed and fresh cell culture medium was added. Hoechst-stained HeLa cells were imaged using a spinning disk confocal microscope. NIS Elements software (Nikon, Japan) was applied for imaging. Hoechst 33342 staining and FD10 were excited using a 408 nm (Hoechst 33342) and 488 nm (FD10) laser line, while Cy5-dextran-NG were excited using a 633 nm laser line. Images with different laser lines were taken in rapid succession with a 0.2 s delay. Hoechst 33342 staining was used to image FITC fluorescence at the focal plane of the cell nucleus. Nuclei were detected in the blue channel and used to determine nuclear FITC fluorescence intensity levels in the green channel as previously described, using ImageJ (FIJI, Version 1.8.0) software.

The amount (number) of Cy5-dex-NG containing endosomes was manually counted in the red channel (Cy5) using thresholding (applying equal offset values for each image). Offset values were normalized to the total cell area, which was determined in the green channel based on FITC fluorescence intensity levels using thresholding. The same threshold settings were maintained for each image. The extent of nanogel uptake was measured in the red channel based on red fluorescence intensity values (mean gray value). These endosomal parameters measured were plotted against the respective nuclear FITC levels for each individual cell for a minimum of 50 cells in total. Simple linear regression analysis was performed to investigate the relationship between FITC-dextran delivery (rMFI FITC) and both endosomal parameters using Graphpad Prism software.

### Quantification of Cre Recombinase and Granzyme a Delivery in HeLa Cells Using Flow Cytometry

Hela cells were seeded at 1×10⁴ cells per well in μ-Slide Angiogenesis Glass Bottom coverslip (ibidi, Munich, Germany). After 24 h, cells were washed twice using PBS. Next, cells were incubated for 2 h in Opti-MEM containing dextran nanogels (dex-NG DS 5.9) together with 5 U Cre recombinase or 10 μg/ml human recombinant granzyme A recombinase in a total volume of 20 μl. Next, excess dex-NG and protein were washed away using PBS. After 24 h, Cre-recombinase treated cells were visualized using confocal microscopy or analyzed using flow cytometry to determine the percentage of eGFP expressing (eGFP+) cells. One day after granzyme A delivery, cell viability was measured using the CellTiter-Glo® luminescent viability assay. To confirm granzyme A mediated cell death, Annexin V staining was performed according to manufacturer's instructions followed by confocal imaging (408 nm laser line) as previously described.

Nucleofection of HeLa reporter cells was performed using a 4D-Nucleofector™ system and SE Cell Line 4D-Nucleofector™ X kit S (Lonza Cologne, Germany) following the manufacturer's instructions. Briefly, HeLa reporter cells were trypsinized and 1×10⁵ cells were resuspended in nucleofector solution containing 5 U Cre recombinase in a total volume of 20 μl and treated with program CN-114 in 20 μl Nucleocuvette™ Strips (Lonza Cologne, Germany). After treatment, the cells were washed and transferred to a μ-Slide Angiogenesis Glass Bottom coverslip containing cell culture medium. After 24 h, the cells were harvested for flow cytometry analysis. As a comparison, the commercial reagent PULSin (Polyplus Transfection, Strasbourg, France) was used according to the manufacturer's instructions. Briefly, HeLa cells were seeded at 1.5×10⁴ cells per well in a glass bottom 96-well plate (Greiner Bio-One GmbH, Vilvoorde, Belgium). Cre recombinase was complexed at 4 μl PULSin per μg Cre recombinase in a total volume of 20 μl of 20 mM Hepes buffer. Next, HeLa reporter cells were washed with PBS and 20 μl of protein-PULSin mix combined with 80 μl serum-free cell culture medium was added to the cells for 4 h. After 48 h, the HeLa reporter cells were harvested for flow cytometry analysis.

### Dex-NG Mediated FITC-Dextran 10 kDa Delivery in Human T Cells

Human T cells were seeded at 1×10⁶ cells per well in a glass bottom 96-well plate (Greiner Bio-One GmbH, Vilvoorde, Belgium). Next, cells were washed twice using PBS and incubated in Opti-MEM containing dex-NG DS 5.9 and 2 mg/ml FD10 in the presence or absence of 10 mM N-acetyl cysteine (NAC). Incubations were performed for 1 h at 37° C. in a humidified atmosphere containing 5% CO₂ unless otherwise specified. Next, nanocarriers and excess proteins were washed away using PBS. After washing, cells were incubated in the presence of 0.5 μM TO-PRO-3 iodine in complete RPMI. Quantitative analysis of delivery efficiency was performed using flow cytometry on living (i.e. TO-PRO-3 negative) cells (CytoFLEX equipped with CytExpert software; Beckman Coulter, Krefeld, Germany). FlowJo software was used for data analysis. For confocal microscopy, human T cell nuclei were stained using Hoechst 33342 and cell viability was confirmed using CellTrace™ Calcein Red-Orange. Briefly, the cells were washed using PBS and incubated in cell culture medium containing 20 μg/ml Hoechst 33342 for 15 min. Finally, staining solution was removed and fresh cell culture medium was added. The cells were kept at 37° C. in humidified atmosphere with 5% CO₂ until confocal imaging. Nucleofection was performed using a 4D-Nucleofector™ system and P3 Primary Cell 4D-Nucleofector™ kit (Lonza Cologne, Germany) following manufacturer's instructions. Briefly, 1×10⁶ human T cells were resuspended in nucleofector solution containing 2 mg/ml FD 10 kDa and treated with program EO-115 (high functionality) in 20 μl Nucleocuvette™ Strips (Lonza Cologne, Germany). After treatment, cells were washed and transferred to a 96-well plate for further analysis.

### Dex-NG Mediated Gadobutrol Delivery in Human T Cells

Human T cells were seeded at 1×10⁶ cells per well in a glass bottom 96-well plate (Greiner Bio-One GmbH, Vilvoorde, Belgium). Next, cells were washed twice using PBS and incubated in Opti-MEM containing dex-NG DS 5.9 and 100 mM gadobutrol or 100 mM gadobutrol only. Incubations were performed for 1 h at 37° C. in a humidified atmosphere containing 5% CO₂. Next, nanogels and excess gadobutrol were washed away by large volumes of PBS. Afterwards, 4×10⁵ human T cells per condition were resuspended in 25 μl and transferred to an 18-well, flat μ-Slide (ibidi, Munich, Germany) for further analysis. As a comparison with the current gold standard for cargo delivery in human T cells, gadobutrol was delivered in human T cells using nucleofection as indicated above. Image acquisition was performed by placing each μ-Slide in the cavity of a 50 ml centrifuge tube containing 1% agar (Invitrogen, Merelbeke, Belgium). Next, a horizontal bore 7 T magnet (PharmaScan, Bruker BioSpin, Ettlingen, Germany) with a mouse whole body volume coil (40 mm inner diameter) was used to acquire MR images. An anatomical scan was taken to obtain spatial information using a spin echo RARE sequence with the following parameters: TR/TE 1730/11.1 ms, RARE factor 4, FOV 4 cm×2.5 cm, matrix 333×208, slice thickness 600 μm, 3 averages, acquisition time 3 min 23 s. R1 relaxometry was performed on a single coronal slice using the following parameters: 10 TRs (8000 ms, 4000 ms, 2000 ms, 1000 ms, 700 ms, 400 ms, 200 ms, 120 ms, 80 ms, 61 ms), TE 11 ms, RARE factor 2, FOV 3 cm×2 cm, matrix 192×128, slice thickness 1 mm, 2 averages, acquisition time 39 min 45 s. Next, R1 values (1/T1) were calculated using the “evolution” script (ParaVision Version 5.1, Bruker BioSpin, Ettlingen, Germany). The total acquisition time was approximately 40 min.

### Statistical Analysis

Statistical analysis was performed using GraphPad Prism software (Version 6). A student t-test was used to compare the mean of 2 conditions. Multiple conditions were compared using a One-way ANOVA combined with the post-hoc Tukey test to correct for multiple testing. When comparing several means to a single control mean a post-hoc Dunnett test was applied. Simple linear regression analysis was performed in the same software. Goodness-of-fit was represented as R². P-values<0.05 were considered to be statistically significant.

### Results and Discussion

### Screening Polycationic Materials for Cytosolic Delivery of Macromolecules

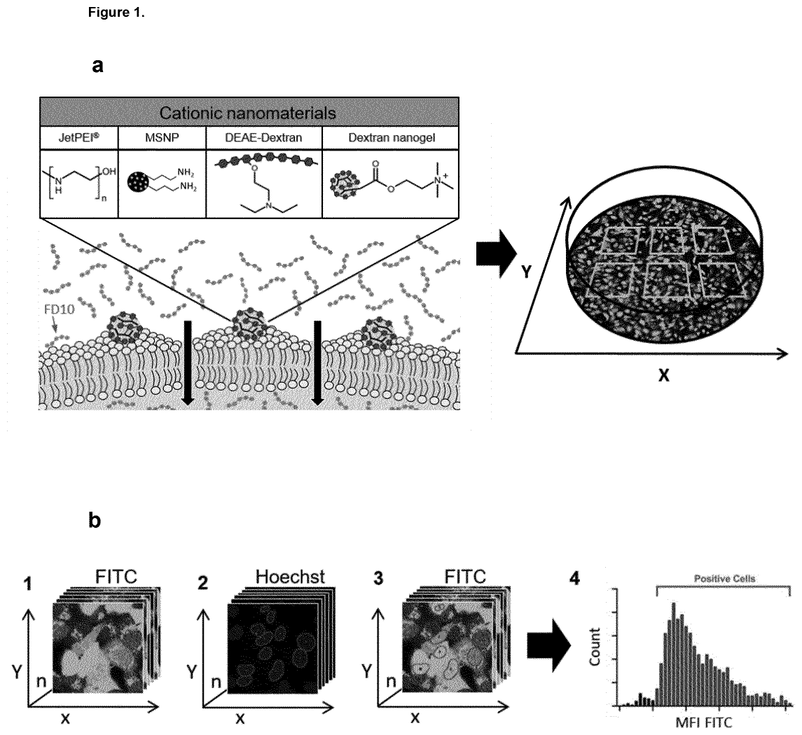

Polycationic materials have been shown to induce lipid membrane defects, including the formation of nanosized pores. To investigate whether these membrane defects could be used for the direct cytosolic entry of membrane-impermeable macromolecules, we tested four commonly used polycationic materials for which the induction of membrane perturbations has been described in literature (7, 10, 11), i.e. two cationic polymers (linear polyethyleneimine (JetPEI®) and diethylaminoethyl (DEAE)-dextran) and two cationic nanoparticles (propylamine-functionalized mesoporous silica nanoparticles (MSNP) and a cationic dextran hydrogel nanoparticle (dextran nanogel, NG)). The experimental procedure to quantify cytosolic delivery is illustrated in FIG. 1a-b. First, HeLa cells were exposed to combinations of the indicated polycationic material at subtoxic concentrations (cell viability ±80%) and neutral FITC-labeled dextran with an average molecular weight of 10 kDa (FD10), used as a model macromolecule. As (fluid phase) endocytosis of cargo typically leads to its endo-lysosomal sequestration, we adopted a quantification method that allows us to distinguish effective cytosolic FD10 delivery from endocytic uptake. Since cytosolic FD10 can diffuse into the nucleus, FD10 delivery efficiency was determined based on fluorescence intensity levels measured in the nuclear region of each individual cell using rapid spinning disk confocal imaging. As such, possible interference arising from endo-lysosomal FD10 fluorescence can be avoided.

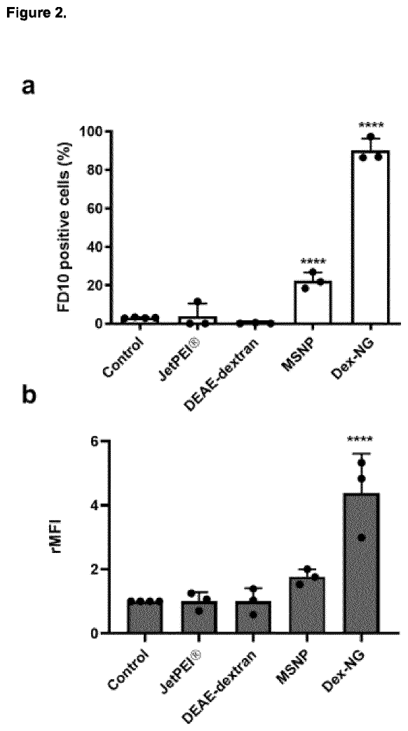

Bright green punctae are visible in HeLa cells exposed to FD10, indicative of endosomal entrapment following spontaneous pinocytic uptake. In addition, these punctae are more pronounced following co-incubation with cationic nanoparticles, possibly demonstrating increased endocytic uptake as a result of nanoparticle-membrane binding. In strong contrast to the cationic polymers JetPEI® and DEAE-dextran, only exposure of HeLa cells to the cationic nanoparticles (MSNPs and dextran nanogels) also caused a marked diffuse staining of the cell cytoplasm and nucleus, indicating successful cytosolic FD10 delivery. However, upon quantifying nuclear FITC fluorescence intensity, dextran nanogels emerged as a far superior polycationic material for cytosolic delivery, showing significantly higher FD10 delivery efficiency (˜90% positive cells, with a relative mean fluorescence intensity (rMFI) of ˜5) compared to MSNPs (˜20% positive cells, ˜1.7 rMFI) (FIG. 2a-b). These data suggest marked differences in cell membrane interactions between soluble cationic polymers and cationic nanoparticles. Moreover, these results indicate that membrane destabilizations caused by cationic nanoparticles can indeed be exploited for the cytosolic delivery of membrane-impermeable macromolecules and that the delivery efficiency can strongly differ between nanoparticle types. Given their relatively high extent of delivery, dextran nanogels were selected to further explore their delivery potential.

### Scrutinizing Nanogel Properties for Improved Cytosolic Cargo Delivery

To promote the cytosolic delivery efficiency of the dextran nanogels, we investigated the impact of several physicochemical parameters reported to influence polycation-mediated membrane disruption. Cationic dextran nanogels (˜200 nm) are synthesized by copolymerizing methacrylated dextran (dex-MA) with a cationic methacrylate monomer (i.e. [2-(methacryloyloxy)ethyl]-trimethylammonium chloride; TMAEMA) using a mini-emulsion UV polymerization technique (12). Through the use of dextrans with varying degrees of methacrylate substitution (DS), defined as the amount of methacrylate groups per 100 glucopyranose residues, nanogels with different crosslink densities and network pore sizes can be obtained (12-16). To assess the influence of hydrogel crosslink density on the cytosolic delivery efficiency of FD10, we synthesized three nanogel types using methacrylated dextrans with mounting DS values (dex-NG DS 3.4, dex-NG DS 4.7 and dex-NG DS 5.9), while keeping both nanogel size (˜200 nm) and zeta potential (˜+21 mV) constant (Table 1).

TABLE 1
Hydrodynamic diameters and zeta potential of the cationic
nanoparticles used for FD10 delivery. Hydrodynamic diameters
and zeta potentials are given as measured by dynamic light
scattering. Values are shown as mean ± SD of three technical
repeats. Dex-HEMA-NG: dextran hydroxyethylmethacrylate (HEMA)-
co-TMAEMA nanogels; Dex-NG DS 3.4: dextran methacrylate (MA)-
co-TMAEMA nanogels with a degree of substitution of 3.4; Dex-
MA-NG +21 mV: Dex-MA-NG with a degree of substitution of
5.9 and a positive zeta potential of ±21 mV; MSNP: propylamine-
functionalized silica nanoparticle.

| | Hydrodynamic | Zeta potential |
|---|---|---|
| Nanocarrier | diameter (nm) ± SD | (mV) ± SD |
| Dex-HEMA-NG | 209 ± 7 | 21.2 ± 0.3 |
| Dex-NG DS 3.4 | 199 ± 7 | 20.6 ± 0.4 |
| Dex-NG DS 4.7 | 198 ± 3 | 21.3 ± 0.9 |
| Dex-NG DS 5.9* | 200 ± 5 | 21.1 ± 0.7 |
| Dex-NG +16 mV | 208 ± 9 | 16.2 ± 0.3 |
| Dex-NG +10 mV | 204 ± 7 | 9.9 ± 0.4 |
| MSNP | 365 ± 11 | 19.5 ± 1.0 |

*identical to Dex-NG +21 mV.

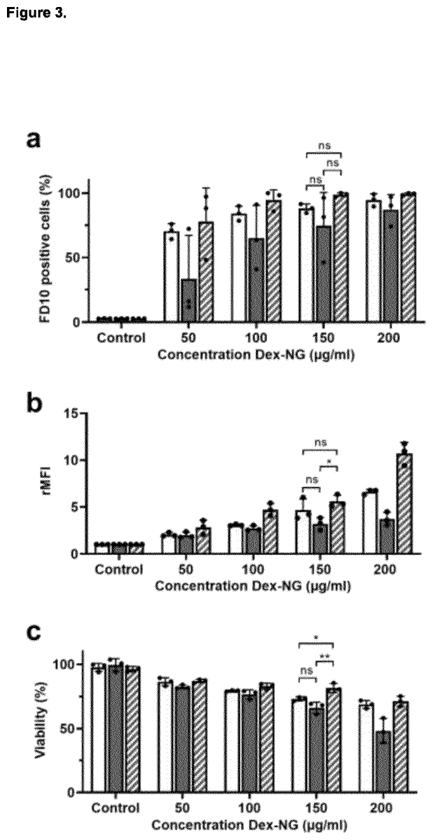

As demonstrated in FIG. 3, all synthesized nanogels enable high cytosolic FD10 delivery in HeLa cells in a dose-dependent manner. Of note, when correlating delivery efficiency with cell viability, dextran nanogels with the highest DS value seem to perform best, with near 100% positive cells while maintaining high cell viability (>80%) at an optimal nanogel concentration of 150 μg/ml. On the other hand, the nanogels with intermediate DS 4.7 display both the lowest FD10 delivery and cell viability. Higher crosslink densities not only result in increased stiffness of the hydrogel network, but also correspond with a higher fraction of hydrophobic methacrylate moieties. While nanoparticle hydrophobicity is positively correlated with membrane destabilization, the inverse relation has been described for particle rigidity. For example, Liechty et al. recently demonstrated that incorporating hydrophobic moieties into a hydrogel network could significantly alter its cell membrane disruption properties (17). This discrepancy could explain why no clear linear correlation between the DS value and FD10 delivery efficiency was observed.

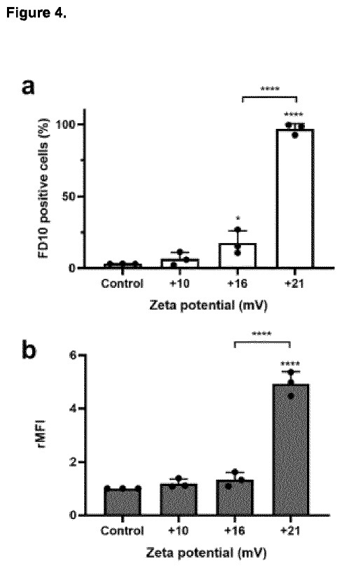

For many polycationic materials, cationic charge density has been consistently reported as a major predictor for induced membrane defects (8). To investigate the effect of cationic charge, three additional dextran nanogels were synthesized by incorporating different fractions of the cationic methacrylate TMAEMA into the dex-MA DS 5.9 hydrogel network. Stable nanogels could be obtained with a zeta-potential of +10 mV, +16 mV and +21 mV, while again maintaining a nanogel size of ˜200 nm (Table 1). In contrast to the DS factor, a clear impact of surface charge on FD10 delivery efficiency was observed, as only nanogels with a sufficiently high cationic charge (i.e. zeta potential of +21 mV) could successfully deliver FD into the cytosol of HeLa cells (FIG. 4a-b). As the charge density can influence both the number of cationic nanogels adhering to the negatively charged cell membrane as well as the interaction strength of individual nanogels with specific cell membrane components, it remains unclear which of these processes is responsible for the observed difference in delivery efficiency. Nonetheless, given the high delivery efficiencies reported for dextran nanogels with a zeta potential of +21 mV and a DS of 5.9, this nanogel formulation was selected to further explore the mechanisms involved in nanogel-mediated membrane disruption.

### A Crosslinked Hydrogel Network is Required for Cytosolic Macromolecule Delivery

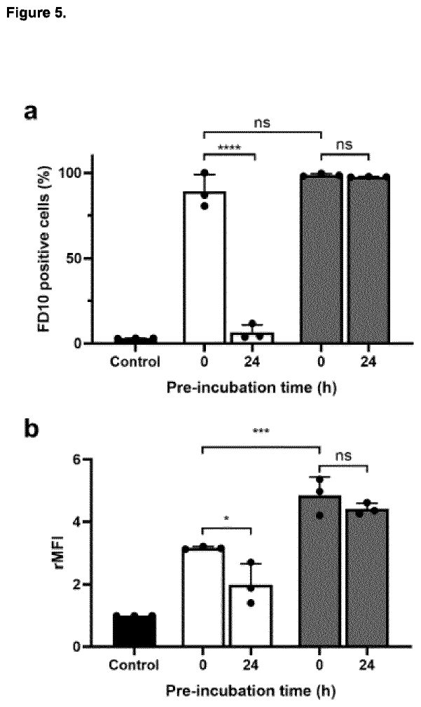

Both cationic nanocarriers used in our initial screen (i.e. MSNPs and dextran nanogels) have been shown to induce membrane perturbations in living cells. Interestingly, our results suggest that only (spherical) nanoparticles and not linear polymers were able to provoke membrane defects large enough for the passage of FD10 (FIG. 2). To further confirm whether an intact hydrogel network was required for dextran nanogel-mediated delivery, we synthesized a degradable cationic dextran nanogel (dex-HEMA DS 5.2 nanogel), composed of dextran chains substituted with hydroxyethyl methacrylate (dex-HEMA), leading to labile crosslinks with a carbonate ester moiety that hydrolyzes in an aqueous environment (16). In contrast, dex-NG (dextran methacrylate-co-TMAEMA nanogels) do not contain these degradable crosslinks and retain a stable 3D network over time (FIG. 10) (12). As can be seen in FIG. 5, dex-HEMA DS 5.2 nanogels (dex-HEMA-NG, size ˜200 nm, zeta-potential ˜+21 mV, Table 1) can successfully deliver FD10 into HeLa cells upon co-incubation at subtoxic concentrations (cell viability >80%), albeit with significantly lower efficiency relative to the previously optimized dex-NG DS 5.9 nanogel. Remarkably and importantly, degrading the dex-HEMA-NG (24 h incubation at 37° C.) prior to administration to HeLa cells completely abolished cytosolic FD10 delivery, in strong contrast to their stable dex-MA counterpart (dex-NG) exposed to equal experimental conditions. These results confirm the need for an intact 3D network for efficient dextran nanogel-mediated cytosolic FD10 delivery. Hydrolysis of the crosslinks in dex-HEMA-co-TMAEMA nanogels will lead to the release of neutral dextran chains and charged linear HEMA-co-TMAEMA oligomers, which do not evoke membrane perturbation. These data further corroborate the observed differences in delivery efficiency between linear cationic polymers and cationic nanoparticles (FIG. 2). Importantly, these data additionally suggest that transient membrane disruption with degradable nanogels can be envisioned, in which the membrane destabilizing effect is gradually lost as a function of hydrogel degradation kinetics. Moreover, ex vivo degradation of the nanogels could facilitate their removal from the final product prior to in vivo administration to patients, which is expected to contribute to the overall safety of the approach.

### Dextran Nanogels Successfully Deliver FITC-Dextran Molecules of Up to 40 kDa into the Cytosol

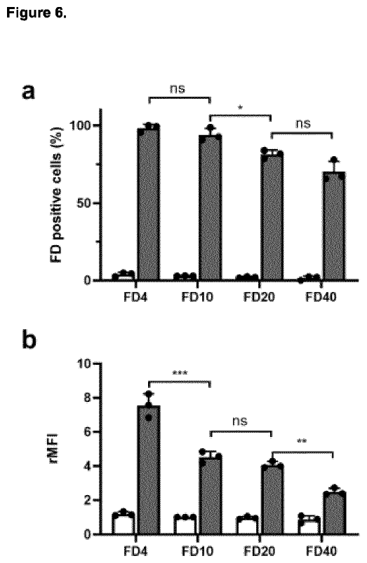

Next, we aimed to investigate to which extent the membrane destabilizations created by dextran-methacrylate nanogels (dex-NG) can be used to deliver larger molecules by incubating the HeLa cells with the nanogels in the presence of FITC-labeled dextrans with varying molecular weight (i.e. FD 4 kDa, FD 10 kDa, FD 20 kDa and FD 40 kDa) (FIG. 6). A clear correlation between the FD size and cytosolic delivery efficiency was seen both for the percentage of positive cells and the measured FITC rMFI in the cell nucleus. A FD molecular weight of 40 kDa (FD40) still resulted in ±70% of positive cells (FIG. 6a), indicating that the delivery efficiency is only moderately reduced with increasing FD molecular weight. However, the decrease in rMFI as a function of FD molecular weight is more outspoken (FIG. 6b). This most likely results from a progressively lower fraction of sufficiently large, induced nanoscale defects able to mediate the entry of molecules higher in molecular weight (40 kDa and higher). Given that FD40 has a reported average diameter of 9 nm, these data are still within the range (15-40 nm) of previously described dimensions of nanoscale membrane pores caused by polycationic materials (11).

### Dextran Nanogel-Mediated Cytosolic Macromolecule Delivery is Endocytosis-Independent

Dex-NG DS 5.9 nanogels are known to be taken up by endocytosis (12). In addition, an increase in endocytic FD10 uptake was seen when co-incubated with cationic nanogels (FIG. 2-6). Therefore, it is conceivable that the observed cytosolic FD delivery could occur through nanogel-induced permeabilization of the plasma membrane, the endosomal membrane or both. To investigate the role of endocytic uptake and subsequent endosomal escape in the cytosolic FD10 delivery process, dex-NG nanogels were first loaded with Cy5-labeled RNA to allow their visualization inside cells via confocal fluorescence microscopy. RNA-loading was performed at an optimized loading ratio as to not interfere with the dex-NG mediated FD delivery process. Such a dual labeling approach allows to correlate endocytic uptake, as measured from the endosomal Cy5 signal, with the cytosolic FD delivery efficiency, for which the nuclear rMFI is a proxy.

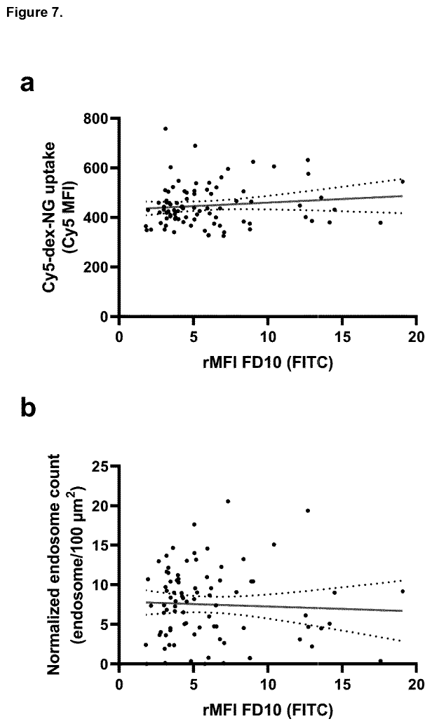

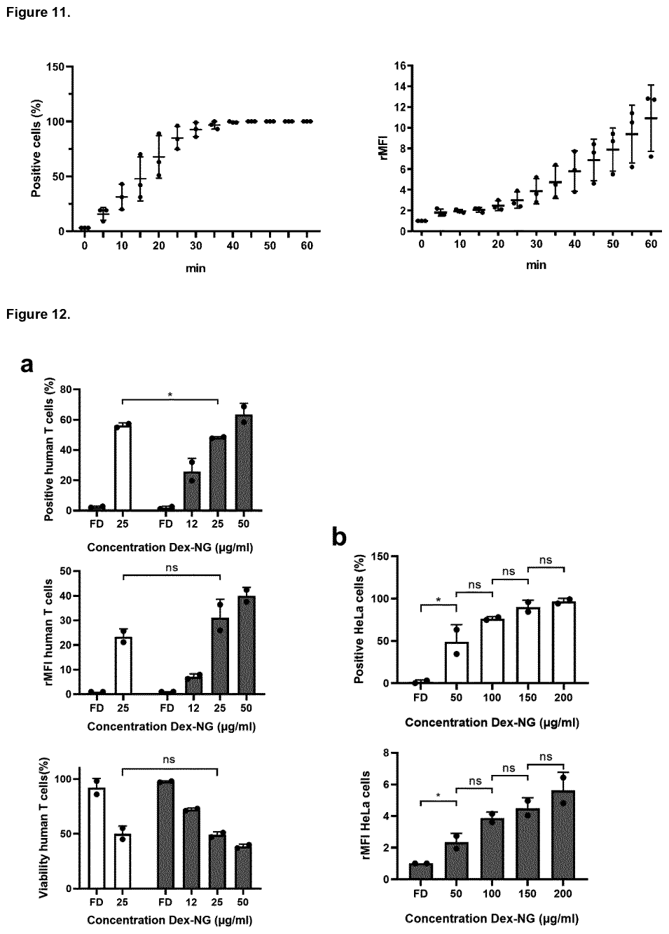

Endocytic uptake was quantified as the total nanogel fluorescence (Cy5 MFI) for each individual cell (FIG. 7a) as well as the total number of nanogel-containing endosomes per cell averaged over the cell area (endosome/100 μm² cell area) to compensate for differences in cell size (FIG. 7b). Using linear regression, no significant correlation could be seen between cytosolic FD delivery and neither total nanogel uptake nor endosome count. In addition, when probing the kinetics of nanogel-induced membrane disruption in HeLa cells, using the membrane-impermeable small molecule nuclear stain propidium iodide (PI), cytosolic delivery was already observed within 5 min (FIG. 11). At 30 min, all cells stained positive for PI after which a linear increase in PI rMFI per cell was observed. Besides demonstrating that this method also enables cytosolic entry of membrane-impermeable small molecules, such fast kinetics of membrane disruption and material influx is in strong contrast with the typically delayed endocytic uptake observed in similarly sized nanoparticles (18-20). Taken together, these data indicate that the nanogel-mediated cytosolic delivery process is not correlated with endocytic uptake and, therefore, likely primarily occurs at the level of the plasma membrane due to pore formation. As such, we termed this newfound delivery platform HyPore, named after its Hydrogel-enabled nanoPoration effect on cell membranes.

### Dextran Nanogels Efficiently Deliver Macromolecules into Primary Human T Cells

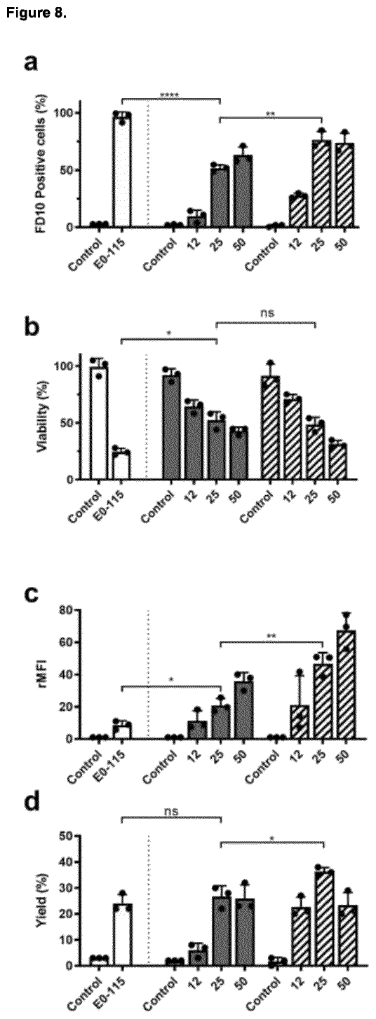

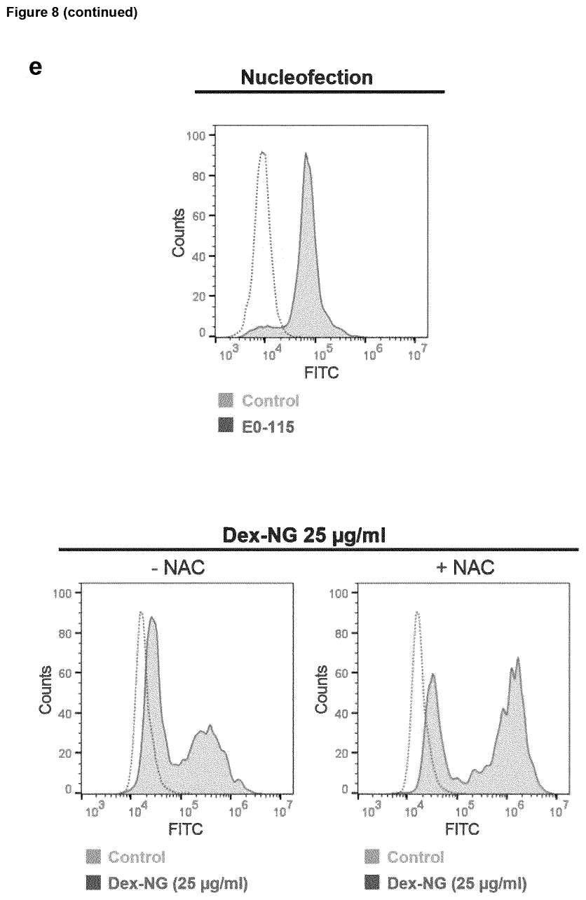

Human T cells are suspension cells that are notoriously hard-to-transfect with conventional carrier-based transfection techniques, in part due to their limited endocytic capacity, thinner cell membrane and relatively low protein content. For these reasons, nucleofection (i.e. an electroporation-based delivery technique) is currently considered the gold standard for the non-viral delivery of macromolecular cargo in these refractory cells. Although we show here that nucleofection can indeed lead to high delivery efficiencies of FD10 in primary human T cells (>95% positive cells, FIG. 8a), it is also associated with substantial loss of cell viability (˜25% remaining cell viability, FIG. 8b). Having established that dextran nanogel (HyPore)-mediated cytosolic delivery was independent of endocytosis, these hydrogel nanoparticles were likewise tested for FD10 delivery in this primary suspension cell type. Optimal delivery was achieved at significantly lower nanogel concentrations compared to adherent HeLa cells (25 μg/ml vs 150 μg/ml for HeLa cells), indicating a clear difference in cell membrane-nanoparticle interactions. HyPore effectively delivered FD10 in >50% of primary T cells, while also maintaining >50% cell viability (FIG. 8a-c). To compare the efficiency of both nucleofection and HyPore-based FD delivery in more detail, we calculated the delivery yield and rMFI of both techniques. The yield is expressed as the percentage of viable cells loaded with cytosolic FD10 and is the product of both the cell viability and the percentage of FD-positive cells. HyPore-mediated delivery realized a comparable delivery yield (28%) relative to nucleofection (22%) (FIG. 8d). Importantly however, the amount of FD10 delivered per cell was about 2.5-fold higher for the HyPore protocol, with an rMFI of 20.9 compared to 8.6 for nucleofection. This marked difference could be explained by the longer incubation time offered by the HyPore delivery platform during which cargo can diffuse into the porated cell. This is in contrast with nucleofection, where generated pores remain open for only a short time (seconds to minutes), thus limiting diffusion-mediated influx. This observed delivery advantage is particularly relevant for T cells given their high cell division rate upon activation. As T cells divide, the cytosolic cargo is diluted over future generations of daughter cells, which can be a limiting factor in certain therapeutic applications as the intracellular titers fall below the minimal effective concentration.

Cellular toxicity induced by polycationic materials, including cationic dextran nanogels, is partially mediated through the formation of reactive oxygen species (ROS), which can be alleviated by the ROS scavenger N-acetylcysteine (NAC)³⁸. In addition, NAC is FDA-approved for various medical uses (e.g. paracetamol overdose, chronic obstructive pulmonary disease) and has recently shown to markedly increase the efficacy of adoptive T cell therapy by improving both T cell mediated tumor control as well as T cell persistence and survival in mice. As such, to further optimize the yield of nanogel-mediated macromolecule delivery in human T cells, NAC was added to the cell medium during nanogel incubation. Unexpectedly, the presence of NAC could not improve T cell viability in our hands (FIG. 8b). In contrast, NAC markedly improved the FD10 delivery efficiency, with increased numbers of FD10-positive cells (>70%) and rMFI values (46.7), further outperforming nucleofection (FIG. 8). This beneficial effect on cytosolic delivery could not be replicated with β-mercaptoethanol, another ROS scavenger commonly used in T cell cultures (FIG. 12). Interestingly, when HeLa cells were treated with a combination of NAC and HyPore, a decrease in FD delivery was observed, suggesting that the delivery-promoting effect of NAC is cell type-dependent.

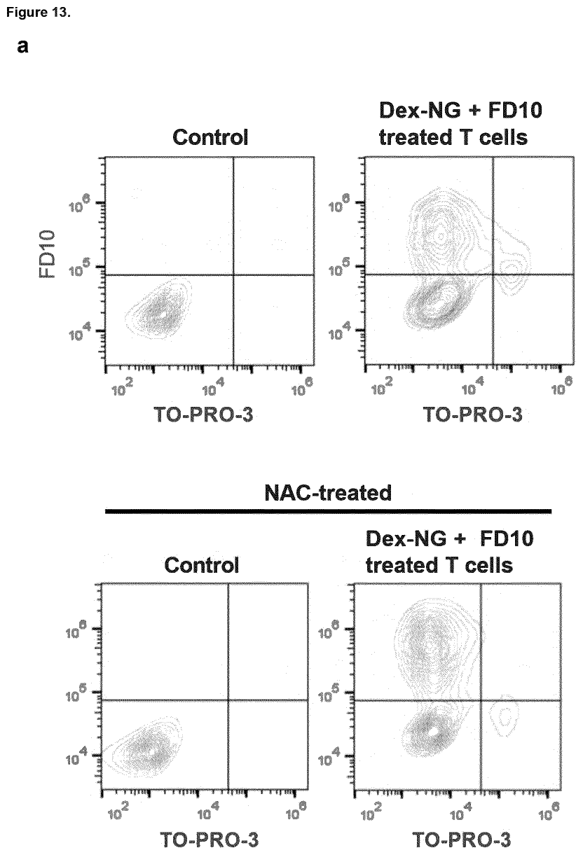

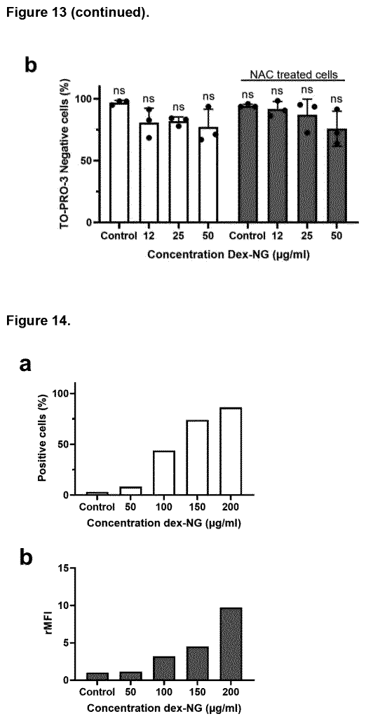

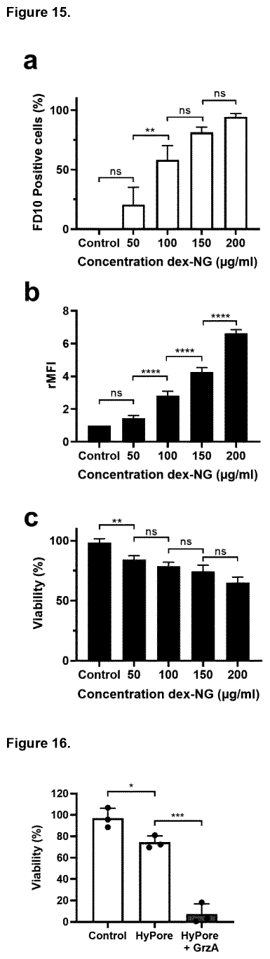

Furthermore, T cell membrane integrity was shown to be quickly restored after nanogel treatment, as human T cells appear impermeable for TO-PRO-3 iodide, after a single wash step following HyPore exposure (FIG. 13). TO-PRO-3 iodide is a cell-impermeable membrane exclusion dye that can only enter cells of which membrane integrity is compromised. These results were also seen in HeLa cells (FIG. 13) and confirm the fast recovery times (˜10⁻¹s) of cationic nanocarrier-induced membrane destabilizations reported in the literature. Finally, in addition to HeLa cells and primary human T cells, dextran nanogels were also able to efficiently deliver FD10 to RAW264.7 cells (murine macrophage cell line) and primary bovine corneal epithelial cells (PBCEC) (FIGS. 14 and 15, respectively). As such, the HyPore delivery platform demonstrates it can be used to deliver cargo to different cell lines as well as hard-to-transfect primary cell lines.

### Dextran Nanogels Allow Intracellular Delivery of Functional Membrane-Impermeable Cargo

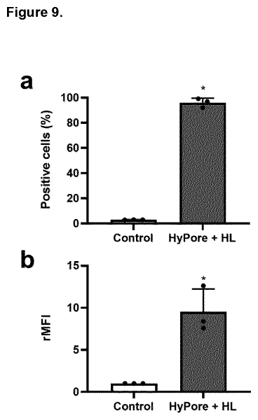

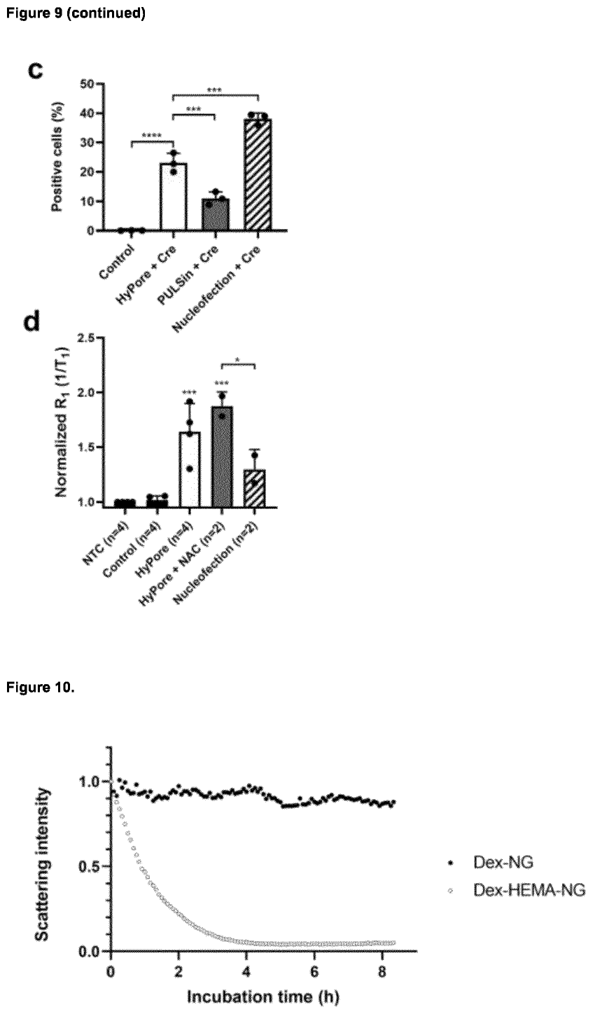

Having established that the HyPore protocol can deliver FDs into a variety of cell types, we next sought to probe the delivery of membrane-impermeable cargo with an intracellular functionality. For instance, intracellular delivery of proteins is of high interest to investigate cellular pathways or to manipulate cells for therapeutic applications. Unfortunately, the development of protein biologics against intracellular targets is hampered by their inability to spontaneously cross cellular membranes. Nanobodies are relatively small single variable-domain antibodies, ˜15 kDa in size, derived from heavy chain only antibodies (HcAbs) typically found in the sera of Camelids. Nanobodies encompass many favorable characteristics compared to conventional full length antibodies, including their small size as well as improved stability and affinity. These specific features rationalize a myriad of biomedical applications, not only as research tools but also as diagnostic and therapeutic agents, e.g. for intracellular applications. Nevertheless, such applications will depend on crossing the cell membrane, hence requiring efficient intracellular delivery approaches. To demonstrate cytosolic nanobody delivery with our HyPore delivery platform, a histone-binding nanobody (Histone-Label, HL), conjugated to the fluorescent dye ATTO488, was used. Cytosolic delivery of HL leads to direct staining of chromosomes and nuclei in cell labeling experiments. As demonstrated in FIG. 9a-b, HyPore-mediated delivery of this fluorescently-labeled nanobody in HeLa cells is remarkably efficient, with over 95% of the cell nuclei successfully stained with a rMFI of 10.8.

As another functional example, we aimed to demonstrate functional cytosolic delivery of enzymes. Granzyme A is a serine protease present in cytotoxic granules of cytotoxic T lymphocytes and natural killer cells. Such cells co-deliver granzymes with perforin, a membranolytic protein that forms pores in endosomal membranes and thus enables cytosolic granzyme delivery in target cells. However, in absence of perforin, granzymes are not able to reach the cytosol. Its delivery to target cells such as tumor cells or viral-infected cells activates a specific caspase-independent cell death pathway. Co-incubation of HeLa cells with HyPore and granzyme A resulted in highly efficient cell killing (FIG. 16). Successful enzyme delivery was further confirmed using confocal microscopy, visualizing the externalization of phosphatidylserine with labeled annexin V, typical for granzyme A-mediated apoptotic cell death. To further demonstrate that HyPore can deliver functional proteins inside cells, we assessed the intracellular delivery of the enzyme Cre recombinase. Cre recombinase is a tyrosine recombinase enzyme derived from the P1 bacteriophages with a size of 38 kDa. Cre binds to a 34 bp long sequence denoted as loxP (locus of crossing (x) over of P1) where it catalyzes a recombination reaction. Its high specificity and efficiency, even when facing complex eukaryotic genomes, explains why even today Cre recombinase remains an important tool for precise and rapid genome editing. Here, we inserted the Cre reporter plasmid pLV-CMV-LoxP-DsRed-LoxP-eGFP in HeLa cells, causing a shift from red (DsRed) to green fluorescence (eGFP) after successful Cre-mediated recombination. Co-incubation of HeLa reporter cells with dextran nanogels and Cre recombinase resulted in 23% of eGFP-expressing HeLa cells (eGFP⁺) (FIG. 9c), compared to only 11% eGFP⁺ cells with PULSin, a commercial delivery reagent for non-invasive protein delivery. As a positive control, nucleofection-mediated delivery resulted in 38% recombination efficiency in these adherent cells. Nonetheless, the ease of use, no requirement for cell detachment or specialized instrumentation, as well as low material cost offer considerable advantages over nucleofection for protein delivery.

Following the high delivery efficiency reported for the HyPore protocol in primary T cells, outperforming nucleofection (FIG. 8), we additionally explored the delivery of the small molecular MRI-contrast agent gadobutrol (Gd-DO3A-butrol), a neutral gadolinium complex that enhances T₁ relaxation (positive contrast) and which can be used for in vivo cell tracking in adoptive cell therapies. As such, the persistence and tissue distribution of adoptively transferred cells can be determined, which is critical to evaluate their immunoregulatory effects in vivo. Nonetheless, the cytosolic delivery of gadobutrol into cells is required as high endosomal gadolinium concentrations following pinocytic uptake have been linked with the quenching of relaxivity. Here, we evaluated the use of HyPore for the cytosolic delivery of the clinically approved gadobutrol into primary human T cells. Nucleofection was selected as a positive control, for which enhanced T1-weighted signals in mammalian cells have been reported upon direct cytosolic gadobutrol delivery. Based on the T₁-weighted images and the calculated relaxation rates, significantly higher signal intensities of HyPore-treated human T cells could be seen compared to cells treated with gadobutrol alone, even at relatively low cell numbers (400 k). Furthermore, NAC and HyPore co-treatment further improved the observed T1-weighted signals, resulting in significantly higher contrast compared to nucleofection-mediated gadobutrol delivery (FIG. 9d).

### CONCLUSION

The present invention demonstrates the use of cationic hydrogel nanoparticles for transient plasma membrane poration and direct cytosolic delivery of membrane-impermeable cargo. This approach merges beneficial aspects of both membrane disruption- and (non-viral) carrier-mediated intracellular delivery techniques. It enables cytosolic delivery of cargo with diverging physicochemical properties in a variety of cell types, including hard-to-transfect cells such as e.g. human primary T cells, without the need for an external physical trigger. Importantly, cytosolic delivery neither requires cargo encapsulation/complexation nor endocytic uptake, thus bypassing the need for endosomal escape and cargo release. Furthermore, these features render HyPore a suitable method for cytosolic delivery of neutral and cationic (macromolecular) compounds, for which state-of-the-art intracellular delivery reagents are not readily available. Finally, HyPore employs relatively simple but flexible materials, which are amenable for upscaling while maintaining low production cost.

### REFERENCES

1. Stewart, M. P.; Langer, R.; Jensen, K. F. Intracellular Delivery by Membrane Disruption: Mechanisms, Strategies, and Concepts. Chem. Rev. 2018, 118, 7409-7531.
2. Stewart, M. P.; Sharei, A.; Ding, X.; Sahay, G.; Langer, R.; Jensen, K. F. In Vitro and Ex Vivo Strategies for Intracellular Delivery. Nature 2016, 538, 183-192.
3. Ramishetti, S.; Kedmi, R.; Goldsmith, M.; Leonard, F.; Sprague, A. G.; Godin, B.; Gozin, M.; Cullis, P. R.; Dykxhoorn, D. M.; Peer, D. Systemic Gene Silencing in Primary T Lymphocytes Using Targeted Lipid Nanoparticles. ACS Nano 2015, 9, 6706-6716.4. Yin, H.; Kanasty, R. L.; Eltoukhy, A. A.; Vegas, A. J.; Dorkin, J. R.; Anderson, D. G. Non-Viral Vectors for Gene-Based Therapy. Nat. Rev. Genet. 2014, 15, 541-555.4. Peer, D.; Karp, J. M.; Hong, S.; Farokhzad, O. C.; Margalit, R.; Langer, R. Nanocarriers as an Emerging Platform for Cancer Therapy. Nat. Nanotechnol. 2007, 2, 751-760.6. Samal, S. K.; Dash, M.; Van Vlierberghe, S.; Kaplan, D. L.; Chiellini, E.; Van Blitterswijk, C.; Moroni, L.; Dubruel, P. Cationic Polymers and Their Therapeutic Potential. Chem. Soc. Rev. 2012, 41, 7147-7149.7. Chen, J.; Hessler, J. A.; Putchakayala, K.; Panama, B. K.; Khan, D. P.; Hong, S.; Mullen, D. G.; DiMaggio, S. C.; Som, A.; Tew, G. N.; et al. Cationic Nanoparticles Induce Nanoscale Disruption in Living Cell Plasma Membranes. J. Phys. Chem. B 2009, 113, 11179-11185.
8. Hong, S.; Leroueil, P. R.; Janus, E. K.; Peters, J. L.; Kober, M. M.; Islam, M. T.; Orr, B. G.; Baker, J. R.; Banaszak Holl, M. M. Interaction of Polycationic Polymers with Supported Lipid Bilayers and Cells: Nanoscale Hole Formation and Enhanced Membrane Permeability. Bioconjug. Chem. 2006, 17, 728-734.
9. Hong, S.; Bielinska, A. U.; Mecke, A.; Keszler, B.; Beals, J. L.; Shi, X.; Balogh, L.; Orr, B. G.; Baker, J. R.; Banaszak Holl, M. M. Interaction of Poly(Amidoamine) Dendrimers with Supported Lipid Bilayers and Cells: Hole Formation and the Relation to Transport. Bioconjug. Chem. 2004, 15, 774-782.
10. Li, S.; Malmstadt, N. Deformation and Poration of Lipid Bilayer Membranes by Cationic Nanoparticles. Soft Matter 2013, 9, 4969-4976.
11. Orr, B. G. Cationic Nanoparticles Induce Nanoscale Disruption in Living Cell Plasma Membranes. 2009, 71, 233-236.
12. Raemdonck, K.; Naeye, B.; Buyens, K.; Vandenbroucke, R. E.; Høgset, A.; Demeester, J.; Smedt, S. C. D.; De Smedt, S. C. Biodegradable Dextran Nanogels for RNA Interference: Focusing on Endosomal Escape and Intracellular SiRNA Delivery. Adv. Funct. Mater. 2009, 19, 1406-1415.
13. Kim, S. hee; Chu, C. C. Pore Structure Analysis of Swollen Dextran-Methacrylate Hydrogels by SEM and Mercury Intrusion Porosimetry. J. Biomed. Mater. Res. 2000, 53, 258-266.
14. Raemdonck, K.; Van Thienen, T. G.; Vandenbroucke, R. E.; Sanders, N. N.; Demeester, J.; De Smedt, S. C. Dextran Microgels for Time-Controlled Delivery of SiRNA. Adv. Funct. Mater. 2008, 18, 993-1001.
15. Stenekes, R. J. H.; De Smedt, S. C.; Demeester, J.; Sun, G.; Zhang, Z.; Hennink, W. E.

Pore Sizes in Hydrated Dextran Microspheres. Biomacromolecules 2000, 1, 696-703.
16. Van Tomme, S. R.; Hennink, W. E. Biodegradable Dextran Hydrogels for Protein Delivery Applications. Expert Rev. Med. Devices 2007, 4, 147-164.
17. Liechty, W. B.; Scheuerle, R. L.; Vela Ramirez, J. E.; Peppas, N. A. Uptake and Function of Membrane-Destabilizing Cationic Nanogels for Intracellular Drug Delivery. Bioeng. Transl. Med. 2019, 4, 17-29.18. Rejman, J.; Oberle, V.; Zuhorn, I. S.; Hoekstra, D. Size-Dependent Internalization of Particles via the Pathways of Clathrin- and Caveolae-Mediated Endocytosis. Biochem. J. 2004, 377, 159-169.
19. Zuhorn, I. S.; Kalicharan, R.; Hoekstra, D. Lipoplex-Mediated Transfection of Mammalian Cells Occurs through the Cholesterol-Dependent Clathrin-Mediated Pathway of Endocytosis. J. Biol. Chem. 2002, 277, 18021-18028.
20. Godbey, W. T.; Wu, K. K.; Mikos, A. G. Tracking the Intracellular Path of Poly(Ethylenimine)/DNA Complexes for Gene Delivery. Proc. Natl. Acad. Sci. U.S.A. 1999, 96, 5177-5188.
21. de Jong, O. G.; Murphy, D. E.; Mager, I.; Willms, E.; Garcia-Guerra, A.; Gitz-Francois, J. J.; Lefferts, J.; Gupta, D.; Steenbeek, S. C.; van Rheenen, J.; et al. A CRISPR-Cas9-Based Reporter System for Single-Cell Detection of Extracellular Vesicle-Mediated Functional Transfer of RNA. Nat. Commun. 2020, 11, 1113.

1. A method for delivery of cell-impermeable molecules across a cell membrane, said method comprising contacting cells with supramolecular polycationic materials, in the presence of the cell-impermeable molecules to be delivered across the cell membrane, and characterized in that the cell-impermeable molecules and supramolecular polycationic materials are independently supplied either simultaneous (co-delivery) or sequentially to the cells.

2. The method according to claim 1, wherein the cell-impermeable molecules and supramolecular polycationic materials are sequentially supplied to the cells; in particular the method comprises contacting the cells with supramolecular polycationic materials and subsequently supplying the cell-impermeable molecules to be delivered across the cell membrane.

3. The method according to claims 1 or 2 wherein the cell-impermeable molecules are neutral or cationic cell-impermeable molecules with respect to the supramolecular polycationic materials when supplied to the cell.

4. The method according to any one of claims 1 to 3 wherein the supramolecular polycationic materials are selected from crosslinked dextran nanogels such as dextran methacrylate (MA)-co-TMAEMA nanogels.

5. The method according to claim 4 wherein the crosslinked dextran nanogels have a Degree of Methacrylate Substitution of at least 2.5 and/or a cationic charge with a Zeta potential of at least 11 mV.

6. The method according to any one of the preceding claims, wherein the cells are contacted with the supramolecular polycationic materials and the cell-impermeable molecules in a solution.

7. The method according to claim 6, wherein the solution is a serum free solution.

8. The method according to any one of the preceding claims, wherein the cells are incubated with the supramolecular polycationic materials for at least 5 min.

9. Use of the methods according to any one of the preceding claims for the in vitro or ex vivo manipulation of primary cells and cell lines.

10. The methods according to any one of the preceding claims, for use in medicine; in particular for use in the treatment of skin or corneal disease.

11. Use of crosslinked cationic nanoparticles as defined in any one of claims 1 to 5, in the delivery of cell-impermeable molecules into a cell.

12. Use of the methods according to any one of the preceding claims, without the need of an external physical trigger.

13. A system for delivery of cell-impermeable molecules into the cell cytrosol, said system comprising supramolecular polycationic materials; in particular crosslinked cationic hydrogel nanoparticles; more in particular crosslinked dextran nanogels such as dextran methacrylate (MA)-co-TMAEMA nanogels, and the like, wherein the cell-impermeable molecules are neutral or cationic cell-impermeable molecules with respect to the supramolecular polycationic materials when supplied to the cell.

14. The system according to claim 13, comprising crosslinked cationic hydrogel nanoparticles (in particular crosslinked dextran nanogels) having a cationic charge with a Zeta potential of at least 11 mV, and/or have a Degree of Methacrylate Substitution of at least 2.5.
